(19) United States
(12) Patent Application Publication
(10) Pub. No.: US 2023/0009832 A1
(43) Pub. Date: Jan. 12, 2023

(54) THERMOSTABLE PHYTASE VARIANTS

(71) Applicant: DUPONT NUTRITION BIOSCIENCES APS, Copenhagen K (DK)

(72) Inventors: Lilia Maria Babe, Emerald Hills, CA (US); Trine Christensen, Risskov (DK); Svend Haaning, Stjaer Galten (DK); Rie Mejldal, Østbirk (DK); Igor Nikolaev, Noordwjik (NL); Sina Pricelius, Leiden (NL); Jens Frisbaek Sørensen, Aarhus N (DK); Robin Anton Sorg, Leiden (NL)

(21) Appl. No.: 17/777,939

(22) PCT Filed: Nov. 20, 2020
(86) PCT No.: PCT/US2020/061469

§ 371 Date: May 18, 2022

Related U.S. Application Data
(60) Provisional application No. 62/937,904, filed on Nov. 20, 2019.

(51) Int. Cl. C12N 9/16; A61K 38/46; A23K 10/18; A23K 20/189; A23K 50/30; A23K 50/75; A23K 50/10

(52) U.S. Cl. CPC C12N 9/16; C12Y 301/03026; A61K 38/465; A23K 10/18; A23K 20/189; A23K 50/30; A23K 50/75; A23K 50/10

(57) ABSTRACT

Provided herein, inter alia, are engineered phytase polypeptides and fragments thereof with improved thermotolerance as well as methods for producing and using the same for enhancing animal performance on one or more metrics.

### CROSS-REFERENCE TO RELATED APPLICATIONS

This application claims priority to U.S. Provisional Patent Application No. 62/937,904, filed on Nov. 20, 2019, the disclosure of which is incorporated by reference herein in its entirety.

### FIELD OF THE INVENTION

Provided herein, inter alia, are engineered phytase polypeptides and fragments thereof with improved thermotolerance as well as methods for producing and using the same for enhancing animal performance.

### BACKGROUND

Phytase is the most commonly used exogenous enzyme in feed for monogastric animals. Phytase can reduce the antinutritional effect of phytate and improve the digestibility of phosphorous, calcium, amino acids and energy, as well as reduce the negative impact of inorganic phosphorous excretion to the environment.

Phytate is the major storage form of phosphorus in cereals and legumes. However, monogastric animals such as pig, poultry and fish are not able to efficiently metabolize or absorb phytate (or phytic acid) in their diet and therefore it is excreted, leading to phosphorous pollution in areas of intense livestock production. Moreover, phytic acid also acts as an anti-nutritional agent in monogastric animals by chelating metal agents such as calcium, copper and zinc and forming insoluble complexes with proteins and amino acids in various segments of the digestive tract.

It has long been assumed that non-ruminant animals lack endogenous phytase and are, thus, incapable of utilizing phytate. However, endogenous mucosal phosphatases and bacterial phytases have been described to have activity in the small intestine and caeca of poultry. Maenz, D. D.; Classen, H. L., Phytase activity in the small intestinal brush border membrane of the chicken. Poult Sci 1998, 77, 557-63. Abudabos, A. M., Phytate phosphorus utilization and intestinal phytase activity in laying hens. Italian Journal of Animal Science 2012, 11, e8. Zeller, E.; Schollenberger, M.; Kuhn, I.; Rodehutscord, M. In order to provide sufficient phosphates for growth and health of these animals, inorganic phosphate is added to their diets. Such addition can be costly and further increases pollution problems.

Through the action of phytase, phytate is generally hydrolysed to give lower inositol-phosphates and inorganic phosphate. Phytases are useful as additives to animal feeds where they improve the availability of organic phosphorus to the animal and decrease phosphate pollution of the environment (Wodzinski R J, Ullah A H. Adv Appl Microbiol. 42, 263-302 (1996)).

A number of phytases of fungal (Wyss M. et al., Appl. Environ. Microbiol. 65 (2), 367-373 (1999); Berka R. M. et al., Appl. Environ. Microbiol. 64 (11), 4423-4427 (1998); Lassen S. et al., Appl. Environ. Microbiol. 67 (10), 4701-4707 (2001)) and bacterial (Greiner R. et al Arch. Biochem. Biophys. 303 (1), 107-113 (1993); Kerovuo et al., Appl. Environ. Microbiol. 64 (6), 2079-2085 (1998); Kim H. W. et al., Biotechnol. Lett. 25, 1231-1234 (2003); Greiner R. et al., Arch. Biochem. Biophys. 341 (2), 201-206 (1997); Yoon S. J. et al., Enzyme and microbial technol. 18, 449-454 (1996); Zinin N. V. et al., FEMS Microbiol. Lett. 236, 283-290 (2004)) origin have been described in the literature.

New generations of phytases have been developed over the last decade. However, none of these phytases has a suitable robustness when applied to feed in a liquid form prior to conditioning and pelleting to withstand the high levels of stress under commercially relevant feed pelleting conditions. Therefore, thermostable phytase products on the market suitable for commercial pelleting are dry products and many have protective coatings to retain activity. However, application of phytases in a liquid form to feed is desirable, because, for example, phytase added in a liquid form will be evenly distributed and immediately released in the animal when delivered via feed. As such, there remains a need for such phytases and fragments thereof which are robust when applied in a liquid form prior to conditioning and pelleting under commercially relevant (i.e. high temperature) conditions and which remain capable of improving animal performance.

The subject matter disclosed herein addresses these needs and provides additional benefits as well.

### SUMMARY

Provided herein, inter alia, are engineered phytase polypeptides and fragments thereof with improved thermotolerance as well as methods for producing and using the same for enhancing animal performance.

Accordingly, in some aspects, provided herein are an engineered phytase polypeptide or a fragment thereof comprising one or more substitutions selected from the group consisting of 30(L, I), 37Y, 45P, 67Y, 89T, 182R, 194M, 202S, 228Y, 256H, 261H, 298V, and 314G, wherein the amino acid positions correspond to SEQ ID NO:1. In some embodiments, the polypeptide or fragment thereof further comprises one or more substitutions selected from the group consisting of 121K, 128N, 131G, 134L, 198Y, 200N, 213Q, 234V, 259E, 270Q, 298V, 320D, 344M, 347Q, and 371T. In some embodiments, the phytase polypeptide or a fragment thereof has at least 80% sequence identity to SEQ ID NO:1. In some embodiments of any of the embodiments disclosed herein, said one or more substitutions increases a) thermostability and/or b) ratio of activity at pH 3.5 versus pH 5.5 compared to phytase polypeptides that lack said one or more substitutions.

In another aspect, provided herein is an engineered phytase polypeptide or a fragment thereof having at least 80% sequence identity to SEQ ID NO:2 and comprising one or more substitutions selected from the group consisting of 30L and 314G, wherein the amino acid positions correspond to SEQ ID NO:2. In some embodiments, the polypeptide or fragment thereof further comprises one or more substitutions selected from the group consisting of 121K, 128N, 134L, 194M, 198Y, 200N, and 270Q. In some embodiments of any of the embodiments disclosed herein, said one or more substitutions increases a) thermostability and/or b) ratio of activity at pH 3.5 versus pH 5.5 compared to phytase polypeptides that lack said one or more substitutions.

In a further aspect, provided herein is an engineered phytase polypeptide or a fragment thereof having at least 80% sequence identity to SEQ ID NO:3, and comprising one or more substitutions selected from the group consisting of 30I, 89T, 182R, 194M, 202S, 228Y, 256H, 261H, and 298V, wherein the amino acid positions correspond to SEQ ID NO:3. In some embodiments, the polypeptide or fragment thereof further comprises one or more substitutions selected from the group consisting of 198Y, 200N, 320D, 347Q, and 371T. In some embodiments of any of the embodiments disclosed herein, said one or more substitutions increases a) thermostability; and/or b) ratio of activity at pH 3.5 versus pH 5.5 compared to phytase polypeptides that lack said one or more substitutions.

In an additional aspect, provided herein is an engineered phytase polypeptide or a fragment thereof having at least 80% sequence identity to SEQ ID NO:4 comprising a 259E substitution, wherein the amino acid position corresponds to SEQ ID NO:4. In some embodiments of any of the embodiments disclosed herein, said one or more substitutions increases a) thermostability, and/or b) ratio of activity at pH 3.5 versus pH 5.5 compared to phytase polypeptides that lack said one or more substitutions.

In yet another aspect, provided herein is an engineered phytase polypeptide or a fragment thereof having at least 80% sequence identity to SEQ ID NO:5 comprising one or more substitutions selected from the group consisting of 30(I/L), 45P, 67Y, and 182R, wherein the amino acid position corresponds to SEQ ID NO:5. In some embodiments, the polypeptide or fragment thereof further comprises a 128N substitution. In some embodiments of any of the embodiments disclosed herein, said one or more substitutions increases a) thermostability; and/or b) ratio of activity at pH 3.5 versus pH 5.5 compared to phytase polypeptides that lack said one or more substitutions.

In another aspect, provided herein is an engineered phytase polypeptide or a fragment thereof having at least 80% sequence identity to SEQ ID NO:6 comprising one or more substitutions selected from the group consisting of 37Y and 45P, wherein the amino acid position corresponds to SEQ ID NO:6. In some embodiments, the polypeptide or fragment thereof further comprises a 131G substitution. In some embodiments of any of the embodiments disclosed herein, said one or more substitutions increases a) thermostability; and/or b) ratio of activity at pH 3.5 versus pH 5.5 compared to phytase polypeptides that lack said one or more substitutions.

In a still further aspect, provided herein is an animal feed, feedstuff, feed additive composition or premix comprising any of the engineered phytase polypeptides or fragments thereof disclosed herein, wherein the engineered phytase polypeptide or fragment thereof may be used (i) alone or (ii) in combination with a direct fed microbial comprising at least one bacterial strain or (iii) with at least one other enzyme or (iv) in combination with a direct fed microbial comprising at least one bacterial strain and at least one other enzyme, or (v) any of (i), (ii), (iii) or (iv) further comprising at least one other feed additive component and, optionally, the engineered phytase polypeptide or fragment thereof is present in an amount of at least about 0.1 g/ton feed.

In another aspect, provided herein is a recombinant construct comprising a regulatory sequence functional in a production host operably linked to a nucleotide sequence encoding any of the engineered phytase polypeptides or fragments thereof disclosed herein. In some embodiments, the production host is selected from the group consisting of bacterial, fungi, yeast, plants and algae.

In an additional aspect, provided herein is a method for producing an engineered phytase polypeptide or fragment thereof comprising: (a) transforming a production host any of the recombinant constructs disclosed herein; and culturing the production host of step (a) under conditions whereby the engineered phytase polypeptide or fragment thereof is produced. In some embodiments, the method further comprises (c) recovering the polypeptide or fragment thereof from the production host.

In some aspects, provided herein is a phytase-containing culture supernatant obtained by the any of the methods for producing an engineered phytase polypeptide or fragment thereof disclosed herein.

In another aspect, provided herein is a polynucleotide sequence encoding any of the engineered phytase polypeptides or fragments thereof disclosed herein.

In yet another aspect, provided herein is a dried enzyme composition for use in animal feed comprising any of the engineered phytase polypeptides or fragments thereof disclosed herein. In some embodiments, the dried enzyme composition is a granulated feed additive composition.

In some aspects, provided herein is a liquid enzyme composition for use in animal feed comprising any of the engineered phytase polypeptides or fragments thereof disclosed herein.

In still further aspects, provided herein is a method for improving the nutritional value of an animal feed, wherein any of the engineered phytase polypeptides or fragments thereof disclosed herein is added to animal feed.

In another aspect, provided herein is a method for improving animal performance on one or more metrics comprising administering an effective amount of a) any of the engineered phytase polypeptides or fragments thereof disclosed herein; or b) any of the animal feed, feedstuff, feed additive compositions or premixes disclosed herein to the animal. In some embodiments, the one or more metrics is selected from the group consisting of increased feed efficiency, increased weight gain, reduced feed conversion ratio, improved digestibility of nutrients or energy in a feed, improved nitrogen retention, improved ability to avoid the negative effects of necrotic enteritis, and improved immune response. In some embodiments of any of the embodiments disclosed herein, the animal is a monogastric animal selected from the group consisting of swine and poultry. In some embodiments, the swine is selected from the group consisting of piglets, growing pigs, and sows. In some embodiments, the poultry is selected from the group consisting of turkeys, ducks, chickens, broiler chicks, layers, geese, pheasants, quail, and emus. In some embodiments of any of the embodiments disclosed herein, the animal is a ruminant animal selected from the group consisting of cattle, young calves, goats, sheep, giraffes, bison, moose, elk, yaks, water buffalo, deer, reindeer, caribou, camels, alpacas, llamas.

Each of the aspects and embodiments described herein are capable of being used together, unless excluded either explicitly or clearly from the context of the embodiment or aspect.

Throughout this specification, various patents, patent applications and other types of publications (e.g., journal articles, electronic database entries, etc.) are referenced. The disclosure of all patents, patent applications, and other publications cited herein are hereby incorporated by reference in their entirety for all purposes.

### BRIEF DESCRIPTION OF THE DRAWINGS

FIG. 1 depicts a multiple sequence alignment of parent phytase backbones showing the positions of amino acid substitutions relative to one another.

### DETAILED DESCRIPTION

### I. Definitions

As used herein, “improving one or more metrics in an animal” refers to improvements on measurements relevant to the growth and/or health of an animal (such as a domesticated bird, for example, a chicken), measured by one or more of the following parameters: average daily weight gain (ADG), overall weight, mortality, feed conversion (which includes both feed:gain and gain:feed), feed intake, intestinal health status, decreased feed conversion ratio (FCR), improved gut barrier integrity, reduced mortality, reduced pathogen infection, and reduced pathogen shedding in feces. “An improvement in a metric” or “improved metric” as used herein, refers to an improvement in at least one of the parameters listed under the metrics in an animal definition.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number can be a number which, in the context in which it is presented, provides the substantial equivalent of the specifically recited number. For example, in connection with a numerical value, the term “about” refers to a range of −10% to +10% of the numerical value, unless the term is otherwise specifically defined in context.

As used herein, the singular forms “a,” “an,” and “the” include plural references unless the context clearly dictates otherwise. For example, the term “a compound” or “at least one compound” may include a plurality of compounds, including mixtures thereof. The terms “a,” “an,” “the,” “one or more,” and “at least one,” for example, can be used interchangeably herein.

The term “and/or” and “or” are used interchangeably herein and refer to a specific disclosure of each of the two specified features or components with or without the other. Thus, the term “and/or” as used in a phrase such “A and/or B” herein is intended to include “A and B,” “A or B,” “A” (alone), and “B” alone. Likewise, the term “and/or” as used a phrase such as “A, B and/or C” is intended to encompass each of the following aspects: A, B and C; A, B or C; A or C; A or B; B or C; A and C; A and B; B and C; A (alone); B (alone and C (alone).

It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is also noted that the term “consisting essentially of,” as used herein refers to a composition wherein the component(s) after the term is in the presence of other known component(s) in a total amount that is less than 30% by weight of the total composition and do not contribute to or interferes with the actions or activities of the component(s).

It is further noted that the term “comprising,” as used herein, means including, but not limited to, the component(s) after the term “comprising.” The components) after the term “comprising” are required or mandatory, but the composition comprising the component(s) can further include other non-mandatory or optional component(s).

It is also noted that the term “consisting of,” as used herein, means including, and limited to, the component(s) after the term “consisting of.” The component(s) after the term “consisting of” are therefore required or mandatory, and no other component(s) are present in the composition.

It is intended that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The term “about” as used herein can allow for a degree of variability in a value or range, for example, within 10%, within 5%, or within 1% of a stated value or of a stated limit of a range.

The term “phytase” (myo-inositol hexakisphosphate phosphohydrolase) refers to a class of phosphatase enzymes that catalyzes the hydrolysis of phytic acid (myo-inositol hexakisphosphate or IP6)—an indigestible, organic form of phosphorus that is found in grains and oil seeds—and releases a usable form of inorganic phosphorus.

The terms “animal” and “subject” are used interchangeably herein and refer to any organism belonging to the kingdom Animalia and includes, without limitation, mammals (excluding humans), non-human animals, domestic animals, livestock, farm animals, zoo animals, breeding stock and the like. For example, there can be mentioned all non-ruminant and ruminant animals. In an embodiment, the animal is a non-ruminant, i.e., mono-gastric animal. Examples of mono-gastric animals include, but are not limited to, pigs and swine, such as piglets, growing pigs, sows; poultry such as turkeys, ducks, chicken, broiler chicks, layers; fish such as salmon, trout, tilapia, catfish and carps; and crustaceans such as shrimps and prawns. In a further embodiment, the animal is a ruminant animal including, but not limited to, cattle, young calves, goats, sheep, giraffes, bison, moose, elk, yaks, water buffalo, deer, camels, alpacas, llamas, antelope, pronghorn and nilgai.

The term “Tₘ” is the temperature at which a protein denatures or the free energy of the unfolded and folded states is equal and half of the population is unfolded and the other half is folded. The thermal unfolding behavior of enzymes is typically studied using calorimetry or optical techniques such as circular dichroism, fluorescence or light scattering.

A “feed” means any natural or artificial diet, meal or the like or components of such meals intended or suitable for being eaten, taken in, digested, by a non-human respectively. Preferably term “feed” is used with reference to products that are fed to animals in the rearing of livestock. The terms “feed” and “animal feed” are used interchangeably herein.

A “feed additive” as used herein refers to one or more ingredients, products of substances (e.g., cells), used alone or together, in nutrition (e.g., to improve the quality of a food (e.g., an animal feed), to improve an animal's performance and/or health, and/or to enhance digestibility of a food or materials within a food.

As used herein, the term “food” is used in a broad sense—and covers food and food products in any form for humans as well as food for animals (i.e. a feed). The food or feed may be in the form of a solution or as a solid—depending on the use and/or the mode of application and/or the mode of administration. In some embodiments, the enzymes mentioned herein may be used as—or in the preparation or production of—a food or feed substance.

As used herein the term “food or feed ingredient” includes a formulation, which is or can be added to foods or foodstuff§ and includes formulations which can be used at low levels in a wide variety of products. The food ingredient may be in the firm of a solution or as a solid—depending on the use and/or the mode of application and/or the mode of administration. The enzymes described herein may be used as a food or feed ingredient or in the preparation or production. The enzymes may be—or may not be added to—food supplements. Feed compositions for monogastric animals typically include compositions comprising plant products which contain phytate. Such compositions include, but are not limited to, cornmeal, soybean meal, rapeseed meal, cottonseed meal, maize, wheat, barley and sorghum-based feeds.

As used herein, the term “pelleting” refers to the production of pellets which can be solid, rounded, spherical and cylindrical tablets, particularly feed pellets and solid, extruded animal feed. One example of a known feed pelleting manufacturing process generally includes admixing together food or feed ingredients at least 1 minutes at room temperature, transferring the admixture to a surge bin, conveying the admixture to a steam conditioner (i.e., conditioning), optionally transferring the steam conditioned admixture to an expander, transferring the admixture to the pellet mill or extruder, and finally transferring the pellets into a pellet cooler, (Fairfield, D. 1994, Chapter 10, Pelleting Cost Center. In Feed Manufacturing Technology IV. (McEllhiney, editor), American Feed industry Association, Arlington, Va., pp. 110-139.).

The term “pellet” refers to a composition of animal feed (usually derived from grain) that has been subjected to a heat treatment, such as a steam treatment (i.e., conditioning), and pressed or extruded through a machine, The pellet may incorporate enzyme in the form of a liquid preparation or a dry preparation. The dry preparation may be coated or not coated and may be in the form of a granule. The term “granule” is used for particles composed of enzymes (such as a phytase, for example, any of the engineered phytase: polypeptides disclosed herein) and other chemicals such as salts and sugars, and may be formed using any of a variety of techniques, including fluid bed granulation approaches to form layered granules.

The term “phytase activity” in relation to determination in solid or liquid preparations means 1 FTU (phytase unit) which is defined as the amount of enzyme required to release 1 micromole of inorganic orthophosphate from a 5.0 mM Sodium phytate substrate (from rice) in one minute under the reaction conditions, pH 5.5 at 37° C., which are also defined in the ISO 2009 phytase assay—A standard assay for determining phytase activity found at International Standard ISO/DIS 30024: 1-17, 2009. Alternatively, as used herein one unit of phytase (U) can be defined against an enzyme standardized using the FTU definition under the reaction conditions 25° C., at 5.5 or 3.5 respectively as described in the assay illustrated in Example 2.

The term “differential scanning calorimetry” or “DSC” as used herein is a thermoanalytical technique in which the difference in the amount of heat required to increase the temperature of a sample and reference is measured as a function of temperature. Both the sample and reference are maintained at nearly the same temperature throughout the experiment. Generally, the temperature program for a DSC analysis is designed such that the sample holder temperature increases linearly as a function of time. The reference sample should have a well-defined heat capacity over the range of temperatures to be scanned.

The term “prebiotic” means a non-digestible food ingredient that beneficially affects the host by selectively stimulating the growth and/or the activity of one or a limited number of beneficial bacteria.

The term “direct-fed microbial” (“DFM”) as used herein is source of live (viable) microorganisms that when applied in sufficient numbers can confer a benefit to the recipient thereof, i.e., a probiotic.

The terms “probiotic,” “probiotic culture,” and “DFM” are used interchangeably herein and define live microorganisms (including bacteria or yeasts for example) which, when for example ingested or locally applied in sufficient numbers, beneficially affects the host organism, i.e. by conferring one or more demonstrable health benefits on the host organism such as a health, digestive, and/or performance benefit. Probiotics may improve the microbial balance in one or more mucosal surfaces. For example, the mucosal surface may be the intestine, the urinary tract, the respiratory tract or the skin. The term “probiotic” as used herein also encompasses live microorganisms that can stimulate the beneficial branches of the immune system and at the same time decrease the inflammatory reactions in a mucosal surface, for example the gut. Whilst there are no lower or upper limits for probiotic intake, it has been suggested that at least 10⁶-10¹², preferably at least 10⁶-10¹⁰, preferably 10⁸-10⁹, cfu as a daily dose will be effective to achieve the beneficial health effects in a subject.

The term “CFU” as used herein means “colony forming units” and is a measure of viable cells in which a colony represents an aggregate of cells derived from a single progenitor cell.

The term “isolated” means a substance in a firm or environment that does not occur in nature and does not reflect the extent to which an isolate has been purified, but indicates isolation or separation from a native form or native environment. Non-limiting examples of isolated substances include (1) any non-naturally occurring substance, (2) any substance including, but not limited to, any host cell, enzyme, engineered enzyme, nucleic acid, protein, peptide or cofactor, that is at least partially removed from one or more or all of the naturally occurring constituents with which it is associated in nature; (3) any substance modified by the hand of man relative to that substance found in nature; or (4) any substance modified by increasing the amount of the substance relative to other components with which it is naturally associated. The terms “isolated nucleic acid molecule”, “isolated polynucleotide”, and “isolated nucleic acid fragment” will be used interchangeably and refer to a polymer of RNA or DNA that is single- or double-stranded, optionally containing synthetic, non-natural or altered nucleotide bases. An isolated nucleic acid molecule in the form of a polymer of DNA may be comprised of one or more segments of cDNA, genomic DNA or synthetic DNA.

The terms “purify,” “purified,” and purification mean to make substantially pure or clear from unwanted components, material defilement, admixture or imperfection. For example, as applied to nucleic acids or polypeptides, purification generally denotes a nucleic acid or polypeptide that is essentially free from other components as determined by analytical techniques well known in the art (e.g., a purified polypeptide or polynucleotide forms a discrete band in an electrophoretic gel, chromatographic eluate, and/or a media subjected to density gradient centrifugation). For example, a nucleic acid or polypeptide that gives rise to essentially one band in an electrophoretic gel is “purified.” A purified nucleic acid or polypeptide is at least about 50% pure, usually at least about 60%, about 65%, about 70%, about 75%, about 80%, about 85%, about 90%, about 91%, about 92%, about 93%, about 94%, about 95%, about 96%, about 97%, about 98%, about 99%, about 99.5%, about 99.6%, about 99.7%, about 99.8% or more pure (e.g., percent by weight on a molar basis). In a related sense, a composition is enriched for a molecule when there is a substantial increase in the concentration of the molecule after application of a purification or enrichment technique. The term “enriched” refers to a compound, polypeptide, cell, nucleic acid, amino acid, or other specified material or component that is present in a composition at a relative or absolute concentration that is higher than a starting composition.

The terms “peptides”, “proteins” and “polypeptides are used interchangeably herein and refer to a polymer of amino acids joined together by peptide bonds. A “protein” or “polypeptide” comprises a polymeric sequence of amino acid residues. The single and 3-letter code for amino acids as defined in conformity with the IUPAC-IUB Joint Commission on Biochemical Nomenclature (JCBN) is used throughout this disclosure. The single letter X refers to any of the twenty amino acids. It is also understood that a polypeptide may be coded for by more than one nucleotide sequence due to the degeneracy of the genetic code. Mutations can be named by the one letter code for the parent amino acid, followed by a position number and then the one letter code for the variant amino acid. For example, mutating glycine (G) at position 87 to serine (S) is represented as “G087S” or “G87S”. When describing modifications, a position followed by amino acids listed in parentheses indicates a list of substitutions at that position by any of the listed amino acids. For example, 6(L, I) means position 6 can be substituted with a leucine or isoleucine. At times, in a sequence, a slash (/) is used to define substitutions, e.g. F/V, indicates that the position may have a phenylalanine or valine at that position.

The terms “signal sequence” and “signal peptide” refer to a sequence of amino acid residues that may participate in the secretion or direct transport of the mature or precursor form of a protein. The signal sequence is typically located N-terminal to the precursor or mature protein sequence. The signal sequence may be endogenous or exogenous. A signal sequence is normally absent from the mature protein. A signal sequence is typically cleaved from the protein by a signal peptidase after the protein is transported.

The term “mature” form of a protein, polypeptide, or peptide refers to the functional form of the protein, polypeptide, or enzyme without the signal peptide sequence and propeptide sequence.

The term “wild-type” in reference to an amino acid sequence or nucleic acid sequence indicates that the amino acid sequence or nucleic acid sequence is a native or naturally-occurring sequence. As used herein, the term “naturally-occurring” refers to anything (e.g., proteins, amino acids, or nucleic acid sequences) that is found in nature. Conversely, the term “non-naturally occurring” refers to anything that is not found in nature (e.g., recombinant/engineered nucleic acids and protein sequences produced in the laboratory or modification of the wild-type sequence).

As used herein with regard to amino acid residue positions, “corresponding to” or “corresponds to” or “correspond to” or “corresponds” refers to an amino acid residue at the enumerated position in a protein or peptide, or an amino acid residue that is analogous, homologous, or equivalent to an enumerated residue in a protein or peptide. As used herein, “corresponding region” generally refers to an analogous position in a related protein or a reference protein.

The terms “derived from” and “obtained from” refer to not only a protein produced or producible by a strain of the organism in question, but also a protein encoded by a DNA sequence isolated from such strain and produced in a host organism containing such DNA sequence. Additionally, the term refers to a protein which is encoded by a DNA sequence of synthetic and/or cDNA origin and which has the identifying characteristics of the protein in question.

The term “amino acid” refers to the basic chemical structural unit of a protein or polypeptide. The following abbreviations used herein to identify specific amino acids can be found in Table 2.

TABLE 2
One and Three Letter Amino Acid Abbreviations

| | Three-Letter | One-Letter |
|---|---|---|
| Amino Acid | Abbreviation | Abbreviation |
| Alanine | Ala | A |
| Arginine | Arg | R |
| Asparagine | Asn | N |
| Thermostable serine acid | Asp | D |
| Cysteine | Cys | C |
| Glutamine | Gln | Q |
| Glutamic acid | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |
| Any amino acid or as defined herein | Xaa | X |

It would be recognized by one of ordinary skill in the art that modifications of amino acid sequences disclosed herein can be made while retaining the function associated with the disclosed amino acid sequences. For example, it is well known in the art that alterations in a gene which result in the production of a chemically equivalent amino acid at a given site, but do not affect the functional properties of the encoded protein are common.

The term “codon optimized”, as it refers to genes or coding regions of nucleic acid molecules for transformation of various hosts, refers to the alteration of codons in the gene or coding regions of the nucleic acid molecules to reflect the typical codon usage of the host organism without altering the polypeptide for which the DNA codes.

The term “coding sequence” refers to a nucleotide sequence which codes for a specific amino acid sequence. “Suitable regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters, translation leader sequences, RNA processing site, effector binding sites, and stem-loop structures.

The term “operably linked” refers to the association of nucleic acid sequences on a single nucleic acid molecule so that the function of one is affected by the other. For example, a promoter is operably linked with a coding sequence when it is capable of affecting the expression of that coding sequence, i.e., the coding sequence is under the transcriptional control of the promoter. Coding sequences can be operably linked to regulatory sequences in sense or antisense orientation.

The terms “regulatory sequence” or “control sequence” are used interchangeably herein and refer to a segment of a nucleotide sequence which is capable of increasing or decreasing expression of specific genes within an organism. Examples of regulatory sequences include, but are not limited to, promoters, signal sequence, operators and the like. As noted above, regulatory sequences can be operably linked in sense or antisense orientation to the coding sequence/gene of interest.

“Promoter” or “promoter sequences” refer a regulatory sequence that is involved in binding RNA polymerase to initiate transcription of a gene. The promoter may be an inducible promoter or a constitutive promoter. A preferred promoter used in the invention is Trichoderma reesei cbh1, which is an inducible promoter.

The “3′ non-coding sequences” refer to DNA sequences located downstream of a coding sequence and include sequences encoding regulatory signals capable of affecting mRNA processing or gene expression, such as termination of transcription.

The term “transformation” as used herein refers to the transfer or introduction of a nucleic acid molecule into a host organism. The nucleic acid molecule may be introduced as a linear or circular form of DNA. The nucleic acid molecule may be a plasmid that replicates autonomously, or it may integrate into the genome of a production host. Production hosts containing the transformed nucleic acid are referred to as “transformed” or “recombinant” or “transgenic” organisms or “transformants”.

The terms “recombinant” and “engineered” refer to an artificial combination of two otherwise separated segments of nucleic acid sequences, e.g., by chemical synthesis or by the manipulation of isolated segments of nucleic acids by genetic engineering techniques. For example, DNA in which one or more segments or genes have been inserted, either naturally or by laboratory manipulation, from a different molecule, from another part of the same molecule, or an artificial sequence, resulting in the introduction of a new sequence in a gene and subsequently in an organism. The terms “recombinant”, “transgenic”, “transformed”, “engineered”, “genetically engineered” and “modified for exogenous gene expression” are used interchangeably herein.

The terms “recombinant construct”, “expression construct”, “recombinant expression construct” and “expression cassette” are used interchangeably herein. A recombinant construct comprises an artificial combination of nucleic acid fragments, e.g., regulatory and coding sequences that are not all found together in nature. For example, a construct may comprise regulatory sequences and coding sequences that are derived from different sources, or regulatory sequences and coding sequences derived from the same source, but arranged in a manner different than that found in nature. Such a construct may be used by itself or may be used in conjunction with a vector. If a vector is used, then the choice of vector is dependent upon the method that will be used to transform host cells as is well known to those skilled in the art. For example, a plasmid vector can be used. The skilled artisan is well aware of the genetic elements that must be present on the vector in order to successfully transform, select and propagate host cells. The skilled artisan will also recognize that different independent transformation events may result in different levels and patterns of expression (Jones et al., (1985) EMBO J 4:2411-2418; De Almeida et al., (1989) Mol Gen Genetics 218:78-86), and thus that multiple events are typically screened to obtain lines displaying the desired expression level and pattern. Such screening may be accomplished using standard molecular biological, biochemical, and other assays including Southern analysis of DNA, Northern analysis of mRNA expression, PCR, real time quantitative PCR (qPCR), reverse transcription PCR (RT-PCR), immunoblotting analysis of protein expression, enzyme or activity assays, and/or phenotypic analysis.

The terms “production host”, “host” and “host cell” are used interchangeably herein and refer to any plant, organism, or cell of any plant or organism, whether human or non-human into which a recombinant construct can be stably or transiently introduced to express a gene. This term encompasses any progeny of a parent cell, which is not identical to the parent cell due to mutations that occur during propagation. In other embodiments, the terms “production host”, “production host cell”, “host cell” and “host strains” are used interchangeably herein and mean a suitable host for an expression vector or DNA construct comprising a polynucleotide encoding phytase polypeptide or fragment thereof. The choice of a production host can be selected from the group consisting of bacteria, fungi, yeast, plants and algae. Typically, the choice will depend upon the gene encoding the engineered phytase polypeptide or fragment thereof and its source.

The term “percent identity” is a relationship between two or more polypeptide sequences or two or more polynucleotide sequences, as determined by comparing the sequences. In the art, “identity” also means the degree of sequence relatedness between polypeptide or polynucleotide sequences, as the case may be, as determined by the number of matching nucleotides or amino acids between strings of such sequences. “Identity” and “similarity” can be readily calculated by known methods, including but not limited to those described in: Computational Molecular Biology (Lesk, A. M., ed.) Oxford University Press, NY (1988); Biocomputing: Informatics and Genome Projects (Smith, D. W., ed.) Academic Press, NY (1993); Computer Analysis of Sequence Data, Part I (Griffin, A. M., and Griffin, H. G., eds.) Humana Press, NJ (1994); Sequence Analysis in Molecular Biology (von Heinje, G., ed.) Academic Press (1987); and Sequence Analysis Primer (Gribskov, M. and Devereux, J, eds.) Stockton Press, NY (1991). Methods to determine identity and similarity are codified in publicly available computer programs.

As used herein, “% identity” or percent identity” or “PID” refers to protein sequence identity. Percent identity may be determined using standard techniques known in the art. Useful algorithms include the BLAST algorithms (See, Altschul et al., J Mol Biol, 215:403-410, 1990; and Karlin and Altschul, Proc Natl Acad Sci USA, 90:5873-5787, 1993). The BLAST program uses several search parameters, most of which are set to the default values. The NCBI BLAST algorithm finds the most relevant sequences in terms of biological similarity but is not recommended for query sequences of less than 20 residues (Altschul et al., Nucleic Acids Res, 25:3389-3402, 1997; and Schaffer et al., Nucleic Acids Res, 29:2994-3005, 2001). Exemplary default BLAST parameters for a nucleic acid sequence searches include: Neighboring words threshold=11; E-value cutoff=10; Scoring Matrix=NUC.3.1 (match=1, mismatch=−3); Gap Opening=5; and Gap Extension=2. Exemplary default BLAST parameters for amino acid sequence searches include: Word size=3; E-value cutoff=10; Scoring Matrix=BLOSUM62; Gap Opening=11; and Gap extension=1. A percent (%) amino acid sequence identity value is determined by the number of matching identical residues divided by the total number of residues of the “reference” sequence. BLAST algorithms refer to the “reference” sequence as the “query” sequence.

As used herein, “homologous proteins” or “homologous phytases” refers to proteins that have distinct similarity in primary, secondary, and/or tertiary structure. Protein homology can refer to the similarity in linear amino acid sequence when proteins are aligned. Homologous search of protein sequences can be done using BLASTP and PSI-BLAST from NCBI BLAST with threshold (E-value cut-off) at 0.001. (Altschul S F, Madde T L, Shaffer A A, Zhang J, Zhang Z, Miller W, Lipman D J. Gapped BLAST and PSI BLAST a new generation of protein database search programs. Nucleic Acids Res 1997 Set 1; 25(17)3389-402). Using this information, proteins sequences can be grouped. Sequence alignments and percent identity calculations may be performed using the Megalign program of the LASERGENE bioinformatics computing suite (DNASTAR Inc., Madison, Wis.), the AlignX program of Vector NTI v. 7.0 (Informax, Inc., Bethesda, MD), or the EMBOSS Open Software Suite (EMBL-EBI; Rice et al., Trends in Genetics 16, (6):276-277 (2000)). Multiple alignment of the sequences can be performed using the CLUSTAL method (such as CLUSTALW; for example, version 1.83) of alignment (Higgins and Sharp, CABIOS, 5:151-153 (1989); Higgins et al., Nucleic Acids Res. 22:4673-4680 (1994); and Chenna et al., Nucleic Acids Res 31 (13):3497-500 (2003)), available from the European Molecular Biology Laboratory via the European Bioinformatics Institute) with the default parameters. Suitable parameters for CLUSTALW protein alignments include GAP Existence penalty=15, GAP extension=0.2, matrix=Gonnet (e.g., Gonnet250), protein ENDGAP=−1, protein GAPDIST=4, and KTUPLE=1. In one embodiment, a fast or slow alignment is used with the default settings where a slow alignment. Alternatively, the parameters using the CLUSTALW method (e.g., version 1.83) may be modified to also use KTUPLE=1, GAP PENALTY=10, GAP extension=1, matrix=BLOSUM (e.g., BLOSUM64), WINDOW=5, and TOP DIAGONALS SAVED=5. Alternatively, multiple sequence alignment may be derived using MAFFT alignment from Geneious® version 10.2.4 with default settings, scoring matrix BLOSUM62, gap open penalty 1.53 and offset value 0.123. The MUSCLE program (Robert C. Edgar. MUSCLE: multiple sequence alignment with high accuracy and high throughput Nucl. Acids Res. (2004) 32 (5): 1792-1797) is yet another example of a multiple sequence alignment algorithm.

The term “engineered phytase polypeptide” means that the polypeptide is not naturally occurring and has phytase activity.

It is noted that a “fragment” of the engineered phytase polypeptide is a portion or subsequence of the engineered phytase polypeptide that is capable of functioning like the engineered phytase polypeptide, i.e., it retains phytase activity.

The term “vector” refers to a polynucleotide sequence designed to introduce nucleic acids into one or more cell types. Vectors include, but are not limited to, cloning vectors, expression vectors, shuttle vectors, plasmids, phage particles, cassettes and the like.

An “expression vector” as used herein means a DNA construct comprising a DNA sequence which is operably linked to a suitable control sequence capable of effecting expression of the DNA in a suitable host. Such control sequences may include a promoter to effect transcription, an optional operator sequence to control transcription, a sequence encoding suitable ribosome binding sites on the mRNA, enhancers and sequences which control termination of transcription and translation.

The term “expression”, as used herein, refers to the production of a functional end-product (e.g., an mRNA or a protein) in either precursor or mature form. Expression may also refer to translation of mRNA into a polypeptide. Expression of a gene involves transcription of the gene and translation of the mRNA into a precursor or mature protein. “Mature” protein refers to a post-translationally processed polypeptide; i.e., one from which any signal sequence, pre- or propeptides present in the primary translation product have been removed. “Precursor” protein refers to the primary product of translation of mRNA; i.e., with pre- and propeptides still present. Pre- and propeptides may be but are not limited to intracellular localization signals. “Stable transformation” refers to the transfer of a nucleic acid fragment into a genome of a host organism, including both nuclear and organellar genomes, resulting in genetically stable inheritance. In contrast, “transient transformation” refers to the transfer of a nucleic acid fragment into the nucleus, or DNA-containing organelle, of a host organism resulting in gene expression without integration or stable inheritance.

Unless defined otherwise herein, all technical and scientific terms used herein have the same meaning as commonly understood by one of ordinary skill in the art to which this invention pertains.

Other definitions of terms may appear throughout the specification.

### II. Compositions

A. Engineered Phytase Polypeptides

As those skilled in the art will appreciate, enzymes are fragile proteins always under threat in the harsh environment of the feed mill. Extremes of temperature, pressure, friction, pH and microbial activity can degrade or destroy enzymes added to feed. The stress on enzyme activity strikes mostly during the conditioning and pelleting phases of processing. For example, the feed absorbs most of its thermal energy during conditioning, prior to pelleting. However, passage from the conditioner through the pellet die also heats the feed. Many factors can contribute to temperature rise through the die, such as, feed formulation, die thickness, die speed, die specification (hole size and shape), initial processing temperature, pelleting capacity etc.

Thus, conditions during feed pelleting on an industrial scale may vary. The ability or robustness of an enzyme to withstand these variations in pelleting conditions is very important. One of ordinary skill in the art will appreciate that conditioning temperatures may vary from feed mill to feed mill. Furthermore, local law needs to be considered in determining the conditions under which the pelleting process is carried out. For instance, Danish law requires 81° C. pelleting of feed for poultry (Miljø-og Fødevareministeriet, fødevarestyrelsen, j.nr. 2017-32-31-00378).

Also, higher temperature pelleting conditions may be used in industry to increase pellet quality such as better durability and reduction of fines and to increase pellet press capacity. A need for a robust phytase that when incorporated in feed prior to conditioning and pelleting can produce pellets of consistent activity over a wide range of temperatures above 80° C. therefore exists. This is important both for liquid-applied phytases and for solid-applied phytases as described herein.

Factors beyond conditioning temperature that may influence the actual stress that a feed enzyme may be subject to include, but are not limited to, feed raw materials, geographical location of the feed mill, equipment used, die size, use of pelleting aids, steam control, temperature control and any other commercially relevant pelleting conditions such as the presence of any other exogenous enzymes that modify feed in such a manner so as to reduce pelleting stress.

These stress factors are further compounded by a trend toward high temperature or super conditioning which leads to the application of enzymes in a liquid form applied post-pelleting.

The terms “liquid”, “liquid form” and “liquid preparation” are used interchangeably and mean that an enzyme can be applied in a liquid form to feed in any manner prior to conditioning and pelleting. It is believed that applying a robust engineered phytase polypeptide or fragment thereof to feed in a liquid form is beneficial as compared to applying such a phytase as a coated granule. This coated granule is the current commercial approach to make phytase products suitable for high temperature conditioning and pelleting. Benefits of liquid application of robust enzyme include; 1) the enzyme will start to work immediately after ingestion by an animal since it does not have to be released from the coated granule before it can interact with the feed, 2) there is improved distribution of the enzyme throughout the feed, thus, ensuring a more consistent delivery of the enzyme to the animal which is particularly important for young animals that eat small amounts of feed, 3) even distribution in the feed makes it easier to measure the enzyme in the feed, and 4) in the case of a robust phytase, such as the engineered phytase polypeptides and fragments disclosed herein, it may start to degrade phytate already present in the feed.

The disclosed engineered phytase polypeptides or fragments thereof were derived using a combination of methods and techniques know in the field of protein engineering which include, phylogenetic analysis, site evaluation libraries, combinatorial libraries, high throughput screening and statistical analysis.

In one aspect, the disclosure relates to an engineered phytase polypeptide or a fragment thereof comprising one or more substitutions selected from the group consisting of 30(L, I), 37Y, 45P, 67Y, 89T, 182R, 194M, 202S, 228Y, 256H, 261H, 298V, and 314G, wherein the amino acid positions correspond to SEQ NO:1. The substitutions, in other embodiments, can also include one or more of the following: 121K, 128N, 131G, 134L, 198Y, 200N, 213Q, 234V, 259E, 270Q, 298V, 320D, 344M, 347Q, and 371T. In some embodiments, the phytase polypeptide or a fragment thereof has at least about 80% sequence identity to SEQ ID NO:1. Those skilled in the art will appreciate that at least 80% sequence identity also includes 80%, 81%, 82%, 83%, 84%, 85%, 86%, 87%, 88%, 89% 90%, 91%, 92%, 93% 94%, 95%, 96%, 97%, 98%, 99% or 100%.

In some embodiments, provided herein are:

a) an engineered phytase polypeptide or a fragment thereof having at least about 80% (such as at least about 80%, 81%, 82%, 83%, 84%, 85%, 86%, 87%, 88%, 89%, 90%, 91%, 92%, 93%, 94%, 95%, 96%, 97%, 98%, 99% or 100%) sequence identity to SEQ ID NO:2 and comprising one or more substitutions selected from the group consisting of 30L and 314G, wherein the amino acid positions correspond to SEQ ID NO:2. The substitutions, in other embodiments, can also include one or more of the following: 121K, 128N, 134L, 194M, 198Y, 200N, and 270Q;

b) an engineered phytase polypeptide or a fragment thereof having at least about 80% (such as at least about 80%, 81%, 82%, 83%, 84%, 85%, 86%, 87%, 88%, 89%, 90%, 91%, 92%, 93%, 94%, 95%, 96%, 97%, 98%, 99% or 100%) sequence identity to SEQ ID NO:3 and comprising one or more substitutions selected from the group consisting of 30I, 89T, 182R, 194M, 202S, 228Y, 256H, 261H, and 298V V, wherein the amino acid positions correspond to SEQ ID NO:3. The substitutions, in other embodiments, can also include one or more of the following: 198Y, 200N, 320D, 347Q, and 371T;

c) an engineered phytase polypeptide or a fragment thereof having at least about 80% (such as at least about 80%, 81%, 82%, 83%, 84%, 85%, 86%, 87%, 88%, 89%, 90%, 91%, 92%, 93%, 94%, 95%, 96%, 97%, 98%, 99% or 100%) sequence identity to SEQ ID NO:4 comprising a 259E substitution, wherein the amino acid position corresponds to SEQ ID NO:4;

d) an engineered phytase polypeptide or a fragment thereof having at least about 80% (such as at least about 80%, 81%, 82%, 83%, 84%, 85%, 86%, 87%, 88%, 89%, 90%, 91%, 92%, 93%, 94%, 95%, 96%, 97%, 98%, 99% or 100%) sequence identity to SEQ ID NO:5 comprising one or more substitutions selected from the group consisting of 30(I/L), 45P, 67Y, and 182R, wherein the amino acid position corresponds to SEQ ID NO:5. The substitutions, in other embodiments, can also include a 128N substitution; and/or

e) an engineered phytase polypeptide or a fragment thereof having at least about 80% (such as at least about 80%, 81%, 82%, 83%, 84%, 85%, 86%, 87%, 88%, 89%, 90%, 91%, 93%, 94%, 95%, 96%, 97%, 98%, 99% or 100%) sequence identity to SEQ ID NO:6 comprising one or more substitutions selected from the group consisting of 37Y and 45P, wherein the amino acid position corresponds to SEQ ID NO:6. The substitutions, in other embodiments, can also include a 131G substitution.

Engineered phytase polypeptides or fragments thereof containing one or more amino acid substitutions can exhibit one or more improved or enhanced properties such as, but not limited to, improved thermostability (such as any of about a 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 30%, 35%, 40%, 45%, 50%, 55%, 60%, 65%, 70%, 75%, 80%, 85%, 90%, 95%, or 100% or greater (inclusive of all percentages falling in between these values) improvement in thermostability) or improved activity (e.g. activity at pH 3.5 compared to activity at pH 5.5) (such as any of about a 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 30%, 35%, 40%, 45%, 50%, 55%, 60%, 65%, 70%, 75%, 80%, 85%, 90%, 95%, or 100% or greater (inclusive of all percentages falling in between these values) improvement in activity) compared to phytase polypeptides or fragments thereof that do not comprise said one or more amino acid substitutions.

In yet other aspects, any of the engineered phytase polypeptides or fragments thereof disclosed herein can have one or more substitutions (such as one or more of the substitutions disclosed above) that increase the ratio between the activity of the phytase at pH 3.5 versus pH 5.5. Consequently, in some embodiments, any of the engineered phytase polypeptides or fragments thereof disclosed herein have a ratio of activity at pH 3.5 compared to the activity (e.g., specific activity) at pH 5.5 of greater than or equal to about 1.2 (such as any of about 1.2, 1.3, 1.4, 1.5, 1.6, 1.7, 1.8, 1.9, 2.0 or higher).

B. Production Hosts

In some embodiments, host strains expressing any of the engineered phytase polypeptides or fragments thereof disclosed herein can be filamentous fungal cells. In one embodiment of the invention, “host cell” means both the cells and protoplasts created from the cells of a filamentous fungal strain and particularly a Trichoderma sp. or an Aspergillus sp.

The term “filamentous fungi” refers to all filamentous forms of the subdivision Eumycotina (See, Alexopoulos, C. J. (1962), INTRODUCTORY MYCOLOGY, Wiley, N.Y.). These fungi are characterized by a vegetative mycelium with a cell wall composed of chitin, cellulose, and other complex polysaccharides. The filamentous fungi of the present invention are morphologically, physiologically, and genetically distinct from yeasts. Vegetative growth by filamentous fungi is by hyphal elongation and carbon catabolism is obligatory aerobic. In the present invention, the filamentous fungal parent cell may be a cell of a species of, but not limited to, Trichoderma, (e.g., Trichoderma reesei (previously classified as T. longibrachiatum and currently also known as Hypocrea jecorina), Trichoderma viride, Trichoderma koningii, Trichoderma harzianum); Penicillium sp., Humicola sp. (e.g., Humicola insolens and Humicola grisea); Chrysosporium sp. (e.g., C. lucknowense), Gliocladium sp., Aspergillus sp. (e.g., A. oryzae, A. niger, and A. awamori), Fusarium sp., Neurospora sp., Hypocrea sp., and Emericella sp. (See also, Innis et al., (1985) Sci. 228:21-26).

As used herein, the term “Trichoderma” or “Trichoderma sp.” refer to any fungal genus previously or currently classified as Trichoderma.

An expression cassette can be included in the production host, particularly in the cells of microbial production hosts. The production host cells can be microbial hosts found within the fungal families and which grow over a wide range of temperature, pH values, and solvent tolerances. For example, it is contemplated that any of bacteria, yeast, plants, algae, or fungi such as filamentous fungi, may suitably host the expression vector. Inclusion of the expression cassette in the production host cell may be used to express the protein of interest so that it may reside intracellularly, extracellularly, or a combination of both inside and outside the cell. Extracellular expression renders recovery of the desired protein from a fermentation product more facile than methods for recovery of protein produced by intracellular expression.

Sometimes it is advantageous to delete genes from expression hosts, where the gene deficiency can be cured by an expression vector. Where it is desired to obtain a fungal host cell having one or more inactivated genes known methods may be used (e.g. methods disclosed in U.S. Pat. Nos. 5.246,853, 5,475,101 and WO92/06209). Gene inactivation may be accomplished by complete or partial deletion, by insertional inactivation or by any other means which renders a gene nonfunctional for its intended purpose such that the gene is prevented from expression of a functional protein).

Any gene from a Trichoderma sp. or other filamentous fungal host, which has been cloned can be deleted, for example cbh1, cbh2, egl1 and egl2 genes. In some embodiments, gene deletion may be accomplished by inserting a firm of the desired gene to be inactivated into a plasmid by methods known in the art. The deletion plasmid is then cut at an appropriate restriction enzyme site(s), internal to the desired gene coding region, and the gene coding sequence or part thereof is replaced with a selectable marker. Flanking DNA sequences from the locus of the gene to be deleted (preferably between about 0.5 to 2.0 kb) remain on either side of the marker gene. An appropriate deletion plasmid will generally have unique restriction enzyme sites present therein to enable the fragment containing the deleted gene, including the flanking DNA sequences and the selectable markers gene to be removed as a single linear piece.

Depending upon the host cell used post-transcriptional and/or post-translational modifications may be made. One non-limiting example of a post-transcriptional and/or post-translational modification is “clipping” or “truncation” of a polypeptide. In another instance, this clipping may result in taking a mature phytase polypeptide and further removing N or C-terminal amino acids to generate truncated forms of the phytase that retain enzymatic activity.

Other examples of post-transcriptional or post-translational modifications include, but are not limited to, myristoylation, glycosylation, truncation, lipidation and tyrosine, serine or threonine phosphorylation. The skilled person will appreciate that the type of post-transcriptional or post-translational modifications that a protein may undergo may depend on the host organism in which the protein is expressed.

Further sequence modifications of polypeptides post expression may occur. This includes, but is not limited to, oxidation, deglycosylation, glycation, etc. It is known that glycation can affect the activity of phytase when subjected to incubation with glucose or other reducing sugars especially at temperatures above 30° C. and neutral or alkaline pH. Protein engineering to eliminate Lysine residues can be used to prevent such modification. An example of this can be found in U.S. Pat. No. 8,507,240. For example, yeast expression can result in highly glycosylated polypeptides resulting in an apparent increased molecular weight. Also, WO2013/119470 (incorporated by reference herein) having international publication date Aug. 15, 2013 relates to phytases having increased stability believed to be due to increased glycosylation. The term “glycosylation” as used herein refers to the attachment of glycans to molecules, for example to proteins. Glycosylation may be an enzymatic reaction. The attachment formed may be through covalent bonds. The phrase “highly glycosylated” refers to a molecule such as an enzyme which is glycosylated in many sites and at all or nearly all the available glycosylation sites, for instance N-linked glycosylation sites. Alternatively, or in addition to, the phrase “highly glycosylated” can refer to extensive glycolytic branching (such as, the size and number of glycolytic moieties associated with a particular N-linked glycosylation site) at all or substantially all N-linked glycosylation sites. In some embodiments, the engineered phytase polypeptide is glycosylated at all or substantially all consensus N-linked glycosylation sites (i.e. an NXS/T consensus N-linked glycosylation site). The term “glycan” as used herein refers to a polysaccharide or oligosaccharide, or the carbohydrate section of a glycoconjugate such as a glycoprotein. Glycans may be homo- or heteropolymers of monosaccharide residues. They may be linear or branched molecules. A phytase may have varying degrees of glycosylation. It is known that such glycosylations may improve stability during storage and in applications. Extensive

The activity of any of the engineered phytase polypeptides or fragments thereof disclosed herein can be determined as discussed within and in accordance with any number of ways known in the art.

B. Additional Enzymes

At least one other enzyme (i.e. in addition to any of the engineered phytase polypeptides or fragments thereof disclosed herein) can be included in the feed additive compositions or formulations disclosed herein which can include, but are not limited to, a xylanase, amylase, another phytase, beta-glucanase, and/or a protease.

1. Xylanases

Xylanase is the name given to a class of enzymes that degrade the linear polysaccharide β-1,4-xylan into xylose, thus breaking down hemicellulose, one of the major components of plant cell walls. Xylanases, e.g., endo-β-xylanases (EC 3.2.1.8) hydrolyze the xylan backbone chain.

In one embodiment, the xylanase may be any commercially available xylanase. Suitably the xylanase may be an endo-1,4-P-d-xylanase (classified as E.G. 3.2.1.8) or a 1,4β-xylosidase (classified as E.G. 3.2.1.37). In one embodiment, the disclosure relates to a composition comprising any of the engineered phytase polypeptides or fragments thereof disclosed herein in combination with an endoxylanase, e.g. an endo-1,4-P-d-xylanase, and another enzyme. All E.C. enzyme classifications referred to herein relate to the classifications provided in Enzyme Nomenclature Recommendations (1992) of the nomenclature committee of the International Union of Biochemistry and Molecular Biology—ISBN 0-12-226164-3, which is incorporated herein.

In another embodiment, the xylanase may be a xylanase from Bacillus, Trichodermna, Therinomyces, Aspergillus, Humicola and Penicillium. In still another embodiment, the xylanase may be the xylanase in Axtra XAP® or Avizyme 1502®, both commercially available products from Danisco A/S. In one embodiment, the xylanase may be a mixture of two or more xylanases. In still another embodiment, the xylanase is an endo-1,4-β-xylanase or a 1,4-β-xylosidase.

In one embodiment, the disclosure relates to a composition comprising any of the engineered phytase polypeptides or fragments thereof disclosed herein and a xylanase. In one embodiment, the composition comprises 10-50, 50-100, 100-150, 150-200, 200-250, 250-300, 300-350, 350-400, 400-450, 450-500, 500-550, 550-600, 600-650, 650-700, 700-750, and greater than 750 xylanase units/g of composition.

In one embodiment, the composition comprises 500-1000, 1000-1500, 1500-2000, 2000-2500, 2500-3000, 3000-3500, 3500-4000, 4000-4500, 4500-5000, 5000-5500, 5500-6000, 6000-6500, 6500-7000, 7000-7500, 7500-8000, and greater than 8000 xylanase units/g composition.

It will be understood that one xylanase unit (XU) is the amount of enzyme that releases 0.5 μmol of reducing sugar equivalents (as xylose by the Dinitrosalicylic acid (DNS) assay-reducing sugar method) from an oat-spelt-xylan substrate per min at pH 5.3 and 50° C. (Bailey, et al., Journal of Biotechnology, Volume 23, (3), May 1992, 257-270).

2. Amylases

Amylase is a class of enzymes capable of hydrolysing starch to shorter-chain oligosaccharides, such as maltose. The glucose moiety can then be more easily transferred from maltose to a monoglyceride or glycosylmonoglyceride than from the original starch molecule. The term amylase includes α-amylases (E.G. 3.2.1.1), G4-forming amylases (E.G. 3.2.1.60), β-amylases (E.G. 3.2.1.2) and γ-amylases (E.C. 3.2.1.3). Amylases may be of bacterial or fungal origin, or chemically modified or protein engineered mutants.

In one embodiment, the amylase may be a mixture of two or more amylases. In another embodiment, the amylase may be an amylase, e.g. an α-amylase, from Bacillus licheniformis and an amylase, e.g. an α-amylase, from Bacillus amyloliquefaciens. In one embodiment, the α-amylase may be the α-amylase in Axtra XAP® or Avizyme 1502®, both commercially available products from Danisco A/S. In yet another embodiment, the amylase may be a pepsin resistant α-amylase, such as a pepsin resistant Trichoderma (such as Trichoderma reesei) alpha amylase. A suitably pepsin resistant α-amylase is taught in UK application number 101 1513.7 (which is incorporated herein by reference) and PCT/IB2011/053018 (which is incorporated herein by reference).

It will be understood that one amylase unit (AU) is the amount of enzyme that releases 1 mmol of glucosidic linkages from a water insoluble cross-linked starch polymer substrate per min at pH 6.5 and 37° C. (this may be referred to herein as the assay for determining 1 AU).

In one embodiment, disclosure relates to a composition comprising any of the engineered phytase polypeptides or fragments thereof disclosed herein and an amylase. In one embodiment, disclosure relates to a composition comprising any of the engineered phytase polypeptides or fragments thereof disclosed herein, xylanase and amylase. In one embodiment, the composition comprises 10-50, 50-100, 100-150, 150-200, 200-250, 250-300, 300-350, 350-400, 400-450, 450-500, 500-550, 550-600, 600-650, 650-700, 700-750, and greater than 750 amylase units/g composition.

In one embodiment, the composition comprises 500-1000, 1000-1500, 1500-2000, 2000-2500, 2500-3000, 3000-3500, 3500-4000, 4000-4500, 4500-5000, 5000-5500, 5500-6000, 6000-6500, 6500-7000, 7000-7500, 7500-8000, 8000-8500, 8500-9000, 9000-9500, 9500-10000, 10000-11000, 11000-12000, 12000-13000, 13000-14000, 14000-15000 and greater than 15000 amylase units/g composition.

3. Proteases

The term protease as used herein is synonymous with peptidase or proteinase. The protease may be a subtilisin (E.G. 3.4.21.62) or a bacillolysin (E.G. 3.4.24.28) or an alkaline serine protease (E.G. 3.4.21.x) or a keratinase (E.G. 3.4.X.X). In one embodiment, the protease is a subtilisin. Suitable proteases include those of animal, vegetable or microbial origin.

Chemically modified or protein engineered mutants are also suitable. The protease may be a serine protease or a metalloprotease. e.g., an alkaline microbial protease or a trypsin-like protease. In one embodiment, provided herein are compositions comprising any of the engineered phytase polypeptides or fragments thereof disclosed herein and one or more protease.

Examples of alkaline proteases are subtilisins, especially those derived from Bacillus sp., e.g., subtilisin Novo, subtilisin Carlsberg, subtilisin 309 (see, e.g., U.S. Pat. No. 6,287,841), subtilisin 147, and subtilisin 168 (see, e.g., WO 89/06279). Examples of trypsin-like proteases are trypsin (e.g., of porcine or bovine origin), and Fusarium proteases (see, e.g., WO 89/06270 and WO 94/25583). Examples of useful proteases also include but are not limited to the variants described in WO 92/19729 and WO 98/20115.

In one embodiment, the protease is selected from the group consisting of subtilisin, a bacillolysin, an alkine serine protease, a keratinase, and a Nocardiopsis protease.

It will be understood that one protease unit (PU) is the amount of enzyme that liberates from the substrate (0.6% casein solution) one microgram of phenolic compound (expressed as tyrosine equivalents) in one minute at pH 7.5 (40 mM Na₂PO₄/lactic acid buffer) and 40° C. This may be referred to as the assay for determining 1 PU.

In one embodiment, disclosure relates to a composition comprising any of the engineered phytase polypeptides or fragments thereof disclosed herein and a protease. In another embodiment, disclosure relates to a composition comprising any of the engineered phytase polypeptides or fragments thereof disclosed herein and a xylanase and a protease. In still another embodiment, the disclosure relates to a composition comprising any of the engineered phytase polypeptides or fragments thereof disclosed herein and an amylase and a protease. In yet another embodiment, the disclosure relates to a composition comprising any of the engineered phytase polypeptides or fragments thereof disclosed herein and a xylanase, an amylase and a protease.

In one embodiment, the composition comprises about 10-50, 50-100, 100-150, 150-200, 200-250, 250-300, 300-350, 350-400, 400-450, 450-500, 500-550, 550-600, 600-650, 650-700, 700-750, and greater than 750 protease units/g composition.

In one embodiment, the composition comprises about 500-1000. 1000-1500, 1500-2000, 2000-2500, 2500-3000, 3000-3500, 3500-4000, 4000-4500, 4500-5000, 5000-5500, 5500-6000, 6000-6500, 6500-7000, 7000-7500, 7500-8000, 8000-8500, 8500-9000, 9000-9500, 9500-10000, 10000-11000, 11000-12000, 12000-13000, 13000-14000, 14000-15000 and greater than 15000 protease units/g composition.

C. Direct Fed Microbials

Any of the engineered phytase polypeptides or fragments thereof disclosed herein can be used in combination with a DFM. A DFM can comprise one or more of such microorganisms such as bacterial strains. Categories of DFMs include Bacillus, Lactic Acid Bacteria and Yeasts. Thus, the term DFM encompasses one or more of the following: direct fed bacteria, direct fed yeast, direct fed yeast and combinations thereof.

Bacilli are unique, gram-positive rods that form spores. These spores are very stable and can withstand environmental conditions such as heat, moisture and a range of pH. These spores germinate into active vegetative cells when ingested by an animal and can be used in meal and pelleted diets. Lactic Acid Bacteria are gram-positive cocci that produce lactic acid which are antagonistic to pathogens. Since Lactic Acid Bacteria appear to be somewhat heat-sensitive, they are not used in pelleted diets. Types of Lactic Acid Bacteria include Bifidobacterium, Lactobacillus and Streptococcus.

At least one direct fed microbial (DFM) may comprise at least one viable microorganism such as a viable bacterial strain or a viable yeast or a viable fungi. Preferably, the DFM comprises at least one viable bacteria.

It is possible that the DFM may be a spore forming bacterial strain and hence the term DFM may be comprised of or contain spores, e.g, bacterial spores. Thus, the term “viable microorganism” as used herein may include microbial spores, such as endospores or conidia. Alternatively, the DFM in the feed additive composition described herein may not comprise of or may not contain microbial spores, e.g. endospores or conidia.

The microorganism may be a naturally-occurring microorganism or it may be a transformed microorganism.

A DFM as described herein may comprise microorganisms from one or more of the following genera: Lactobacillus, Lactococcus, Streptococcus, Bacillus, Pediococcus, Enterococcus, Leuconostoc, Carnobacterium, Propionibacterium, Bifidobacterium, Clostridium and Megasphaera and combinations thereof.

In some embodiments, the DFM comprises one or more bacterial strains selected from the following Bacillus spp: Bacillus subtilis, Bacillus cereus, Bacillus licheniformis, Bacillus pumilis and Bacillus amyloliquefaciens.

The genus “Bacillus”, as used herein, includes all species within the genus “Bacillus,” as known to those of skill in the art, including but not limited to B. subtilis, B. licheniformis, B. lentus, B. brevis, B. stearothermophilus, B. alkalophilus, B. amyloliquefaciens, B. clausii, B. halodurans, B. megaterium, B. coagulans, B. circulans, B. gibsonii, B. pumilis and B. thuringiensis. It is recognized that the genus Bacillus continues to undergo taxonomical reorganization. Thus, it is intended that the genus include species that have been reclassified, including but not limited to such organisms as Bacillus stearothermophilus, which is now named “Geobacillus stearothermophilus”, or Bacillus polymyxa, which is now “Paenibacillus polymyxa” The production of resistant endospores under stressful environmental conditions is considered the defining feature of the genus Bacillus, although this characteristic also applies to the recently named Alicyclobacillus, Amphibacillus, Aneurinibacillus, Anoxybacillus, Brevibacillus, Filobacillus, Gracilibacillus, Halobacillus, Paenibacillus, Salibacillus, Thermobacillus, Ureibacillus, and Virgibacillus.

In another aspect, the DFM may be further combined with the following Lactococcus spp: Lactococcus cremoris and Lactococcus lactis and combinations thereof.

The DFM may be further combined with the following Lactobacillus spp: Lactobacillus buchneri, Lactobacillus acidophilus, Lactobacillus casei, Lactobacillus kefiri, Lactobacillus bifidus, Lactobacillus brevis, Lactobacillus helveticus, Lactobacillus paracasei, Lactobacillus rhamnosus, Lactobacillus salivarius, Lactobacillus curvatus, Lactobacillus bulgaricus, Lactobacillus sakei, Lactobacillus reuteri, Lactobacillus fermentum, Lactobacillus farciminis, Lactobacillus lactis, Lactobacillus delbreuckii, Lactobacillus plantarum, Lactobacillus paraplantarum, Lactobacillus farciminis, Lactobacillus rhamnosus, Lactobacillus crispatus, Lactobacillus gasseri, Lactobacillus johnsonii and Lactobacillus jensenii, and combinations of any thereof.

In still another aspect, the DFM may be further combined with the following Bifidobacteria spp: Bifidobacterium lactis, Bifidobacterium bifidium, Bifidobacterium longum, Bifidobacterium animalis, Bifidobacterium breve, Bifidobacterium infantis, Bifidobacterium catenulatum, Bifidobacterium pseudocatenulatum, Bifidobacterium adolescentis, and Bifidobacterium angulatum, and combinations of any thereof.

There can be mentioned bacteria of the following species: Bacillus subtilis, Bacillus licheniformis, Bacillus amyloliquefaciens, Bacillus pumilis, Enterococcus, Enterococcus spp, and Pediococcus spp, Lactobacillus spp, Bifidobacterium spp, Lactobacillus acidophilus, Pediococsus acidilactici, Lactococcus lactis, Bifidobacterium bifidum, Bacillus subtilis, Propionibacterium thoenii, Lactobacillus farciminis, Lactobacillus rhamnosus, Megasphaera elsdenii, Clostridium butyricum, Bifidobacterium animalis ssp. animalis, Lactobacillus reuteri, Bacillus cereus, Lactobacillus salivarius ssp. Salivarius, Propionibacteria sp and combinations thereof.

A direct-fed microbial described herein comprising one or more bacterial strains may be of the same type (genus, species and strain) or may comprise a mixture of genera, species and/or strains.

Alternatively, a DFM may be combined with one or more of the products or the microorganisms contained in those products disclosed in WO2012110778, and summarized as follows: Bacillus subtilis strain 2084 Accession No. NRRLB-50013, Bacillus subtilis strain LSSAO1 Accession No. NRRL B-50104, and Bacillus subtilis strain 15A-P4 ATCC Accession No. PTA-6507 (from Enviva Pro®. (formerly known as Avicorr®); Bacillus subtilis Strain C3102 (from Calsporin®); Bacillus subtilis Strain PB6 (from Clostat®); Bacillus pumilis (8G-134); Enterococcus NCIMB 10415 (SF68) (from Cylactin®); Bacillus subtilis Strain C3102 (from Gallipro® & GalliproMax®); Bacillus licheniformis (from Gallipro®Tect®); Enterococcus and Pediococcus (from Poultry star®); Lactobacillus, Bifidobacterium and/or Enterococcus from Protexin®); Bacillus subtilis strain QST 713 (from Proflora®); Bacillus amyloliquefaciens CECT-5940 (from Ecobiol® & Ecobiol® Plus); Enterococcus faecium SF68 (from Fortiflora®); Bacillus subtilis and Bacillus licheniformis (from BioPlus2B®); Lactic acid bacteria 7 Enterococcus faecium (from Lactiferm®); Bacillus strain (from CSI®); Saccharomyces cerevisiae (from Yea-Sacc®); Enterococcus (from Biomin IMB52®); Pediococcus acidilactici, Enterococcus, Bifidobacterium animalis ssp. animalis, Lactobacillus reuteri, Lactobacillus salivarius ssp. salivarius (from Biomin C5®); Lactobacillus farciminis (from Biacton®); Enterococcus (from Oralin E1707®); Enterococcus (2 strains), Lactococcus lactic DSM 1103 (from Probios-pioneer PDFM®); Lactobacillus rhamnosus and Lactobacillus farciminis (from Sorbiflore®); Bacillus subtilis (from Animavit®); Enterococcus (from Bonvital®); Saccharomyces cerevisiae (from Levucell SB 20®); Saccharomyces cerevisiae (from Levucell SC 0 & SC10® ME); Pediococcus acidilacti (from Bactocell); Saccharomyces cerevisiae (from ActiSaf® (formerly BioSaf®)); Saccharomyces cerevisiae NCYC Sc47 (from Actisaf® SC47); Clostridium butyricum (from Miya-Gold®); Enterococcus (from Fecinor and Fecinor Plus®); Saccharomyces cerevisiae NCYC R-625 (from InteSwine®); Saccharomyces cerevisia (from BioSprint®); Enterococcus and Lactobacillus rhamnosus (from Provita®); Bacillus subtilis and Aspergillus oryzae (from PepSoyGen-C®); Bacillus cereus (from Toyocerin®); Bacillus cereus var. toyoi NCIMB 40112/CNCM I-1012 (from TOYOCERIN®), or other DFMs such as Bacillus licheniformis and Bacillus subtilis (from BioPlus® YC) and Bacillus subtilis (from GalliPro®).

The DFM may be combined with Enviva® PRO which is commercially available from Danisco A/S. Enviva Pro® is a combination of Bacillus strain 2084 Accession No. NRRL B-50013, Bacillus strain LSSAO1 Accession No. NRRL B-50104 and Bacillus strain 15A-P4 ATCC Accession No. PTA-6507 (as taught in U.S. Pat. No. 7,754,469 B—incorporated herein by reference).

It is also possible to combine the DFM described herein with a yeast from the genera: Saccharomyces spp.

Preferably, the DFM described herein comprises microorganisms which are generally recognized as safe (GRAS) and, preferably are GRAS-approved.

A person of ordinary skill in the art will readily be aware of specific species and/or strains of microorganisms from within the genera described herein which are used in the food and/or agricultural industries and which are generally considered suitable for animal consumption.

In some embodiments, it is important that the DFM be heat tolerant, i.e. is thermotolerant. This is particularly the case when the feed is pelleted. Therefore, in another embodiment, the DFM may be a thermotolerant microorganism, such as a thermotolerant bacteria, including for example Bacillus spp.

In other aspects, it may be desirable that the DFM comprises a spore producing bacteria, such as Bacilli, e.g. Bacillus spp. Bacilli are able to form stable endospores when conditions for growth are unfavorable and are very resistant to heat, pH, moisture and disinfectants.

The DFM described herein may decrease or prevent intestinal establishment of pathogenic microorganism (such as Clostridium perfringens and/or E. coli and/or Salmonella spp and/or Campylobacter spp.). In other words, the DFM may be antipathogenic. The term “antipathogenic” as used herein means the DFM counters an effect (negative effects of a pathogen.

Preferably, a representative pathogen used in this DFM assay can be one (or more) of the following: Clostridium, such as Clostridium perfringens and/or Clostridium difficile, and/or E. coli and/or Salmonella spp and/or Campylobacter spp. In one preferred embodiment, the assay is conducted with one or more of Clostridium perfringens and/or Clostridium difficile and/or E. coli, preferably Clostridium perfringens and/or Clostridium difficile, more preferably Clostridium perfringens.

Antipathogenic DFMs include one or more of the following bacteria and are described in WO2013029013. Bacillus subtilis strain 3BP5 Accession No. NRRL B-50510, Bacillus amyloliquefaciens strain 918 ATCC Accession No. NRRL B-50508, and Bacillus amyloliquefaciens strain 1013 ATCC Accession No. NRRL B-50509.

DFMs may be prepared as culture(s) and carrier(s) (where used) and can be added to a ribbon or paddle mixer and mixed for about 15 minutes, although the timing can be increased or decreased. The components are blended such that a uniform mixture of the cultures and carriers result. The final product is preferably a dry, flowable powder. The DFM(s) comprising one or more bacterial strains can then be added to animal feed or a feed premix, added to an animal's water, or administered in other ways known in the art (preferably simultaneously with the enzymes described herein.

Inclusion of the individual strains in the DFM mixture can be in proportions varying from 1% to 99% and, preferably, from 25% to 75%. Suitable dosages of the DFM animal feed may range from about 1×10³ CFU/g feed to about 1×10¹⁰ CFU/g feed, suitably between about 1×10⁴ CFU/g feed to about 1×10⁸ CFU/g feed, suitably between about 7.5×10⁴ CFU/g feed to about 1×10⁷ CFU/g feed. In another aspect, the DFM may be dosed in feedstuff at more than about 1×10³ CFU/g feed, suitably more than about 1×10⁴ CFU/g feed, suitably more than about 5×10⁴ CFU/g feed, or suitably more than about 1×10⁵ CFU/g feed.

The DFM may be dosed in a feed additive composition from about 1×10³ CFU/g composition to about 1×10¹³ CFU/g composition, preferably 1×10⁵ CFU/g composition to about 1×10¹³ CFU/g composition, more preferably between about 1×10⁶ CFU/g composition to about 1×10¹² CFU/g composition, and most preferably between about 3.75×10⁷ CFU/g composition to about 1×10¹¹ CFU/g composition. In another aspect, the DFM may be dosed in a feed additive composition at more than about 1×10⁵ CFU/g composition, preferably more than about 1×10⁶ CFU/g composition, and most preferably more than about 3.75×10⁷ CFU/g composition. In one embodiment, the DFM is dosed in the feed additive composition at more than about 2×10⁵ CFU/g composition, suitably more than about 2×10⁶ CFU/g composition, suitably more than about 3.75×10⁷ CFU/g composition.

E. Feeds and Feedstuffs

In another embodiment, there is described an animal feed, feedstuff, feed additive composition or premix comprising any of the engineered phytase polypeptides or fragments thereof described herein.

Importantly, feed additive enzymes, e.g, a phytase, is subjected to very harsh conditions as it passes through the digestive track of an animal, i.e, low pH and presence of digestive enzymes. Pepsin is one of the most important proteolytic digestive enzymes present in the gastrointestinal tract of monogastric animals. Pepsin has low specificity and high pH tolerance in the acidic area (pH 1.5-6.0 stabile up to pH 8.0). The engineered phytase polypeptides or fragments thereof described herein are largely resistant against pepsin, which is necessary for good in-vivo performance.

The animal feed, feedstuff, feed additive composition or premix comprising any of the engineered phytase polypeptides or fragments thereof described herein may be used (i) alone or (ii) in combination with a direct fed microbial comprising at least one bacterial strain or (iii) with at least one other enzyme or (iv) in combination with a direct fed microbial comprising at least one bacterial strain and at least one other enzyme, or (v) any of (i), (ii), (iii) or (iv) further comprising at least one other feed additive component and, optionally, the engineered phytase polypeptide or fragment thereof is present in an amount of at least 0.1 g/ton feed.

The terms “feed additive”, “feed additive components”, and/or “feed additive ingredients” are used interchangeably herein.

Feed additives can be described as products used in animal nutrition for purposes of improving the quality of feed and the quality of food from animal origin, or to improve the animals' performance and health, e.g. providing enhanced digestibility of the feed materials.

Feed additives fall into a number of categories such as sensory additives which stimulate an animal's appetite so that they naturally want to eat more. Nutritional additives provide a particular nutrient that may be deficient in an animal's diet. Zootechnical additives improve the overall nutritional value of an animal's diet through additives in the feed.

Examples of such teed additives include, but are not limited to, prebiotics, essential oils (such as, without limitation, thymol and/or cinnamaldehyde), fatty acids, short chain fatty acids such as propionic acid and butyric acid, etc., vitamins, minerals, amino acids, etc.

Feed additive compositions or formulations may also comprise at least one component selected from the group consisting of a protein, a peptide, sucrose, lactose, sorbitol, glycerol, propylene glycol, sodium chloride, sodium sulfate, sodium acetate, sodium citrate, sodium formate, sodium sorbate, potassium chloride, potassium sulfate, potassium acetate, potassium citrate, potassium formate, potassium acetate, potassium sorbate, magnesium chloride, magnesium sulfate, magnesium acetate, magnesium citrate, magnesium formate, magnesium sorbate, sodium metabisulfite, methyl paraben and propyl paraben.

In still another aspect, there is disclosed a granulated feed additive composition for use in animal feed comprising at least one polypeptide having phytase activity as described herein, used either alone or in combination with at least one direct fed microbial or in combination with at least one other enzyme or in combination with at least one direct fed microbial and at least one other enzyme, wherein the feed additive composition comprises may be in any form such as a granulated particle. Such granulated particles may be produced by a process selected from the group consisting of high shear granulation, drum granulation, extrusion, spheronization, fluidized bed agglomeration, fluidized bed spray coating, spray drying, freeze drying, milling, spray chilling, spinning disk atomization, coacervation, tableting, or any combination of the above processes.

Furthermore, particles of the granulated feed additive composition can ave a mean diameter of greater than 50 microns and less than 2000 microns

Those skilled in the art will understand that animal feed may include plant material such as corn, wheat, sorghum, soybean, canola, sunflower or mixtures of any of these plant materials or plant protein sources for poultry, pigs, ruminants, aquaculture and pets. It is contemplated that animal performance parameters, such as growth, feed intake and feed efficiency, but also improved uniformity, reduced ammonia concentration in the animal house and consequently improved welfare and health status of the animals will be improved.

### III. Methods

A. Methods for Producing an Engineered Phytase

Also described herein is a method for producing an engineered phytase polypeptide or fragment thereof comprising: (a) transforming a production host (such as any of the production hosts described herein) with the recombinant construct described herein; and (b) culturing the production host of step (a) under conditions whereby the engineered phytase polypeptide or fragment thereof is produced. Optionally, the engineered phytase polypeptide or fragment thereof may be recovered from the production host. In another aspect, a phytase-containing culture supernatant can be obtained by any of the methods disclosed herein.

Thus, in one embodiment, there is described a recombinant construct comprising a regulatory sequence functional in a production host operably linked to a nucleotide sequence encoding an engineered phytase polypeptide and fragments thereof as described herein. This recombinant construct may comprise a regulatory sequence functional in a production host operably linked to a nucleotide sequence encoding any of the engineered phytase polypeptide and fragments thereof described herein. Furthermore, the production host is selected from the group consisting of bacteria, fungi, yeast, plants or algae. The preferred production host is the filamentous fungus, Trichoderma reesei.

In another embodiment, there is described a polynucleotide sequence encoding any of the engineered phytase polypeptides or fragments thereof as described herein. Possible initiation control regions or promoters that can be included in the expression vector are numerous and familiar to those skilled in the art. A “constitutive promoter” is a promoter that is active under most environmental and developmental conditions. An “inducible” or “repressible” promoter is a promoter that is active under environmental or developmental regulation. In some embodiments, promoters are inducible or repressible due to changes in environmental factors including but not limited to, carbon, nitrogen or other nutrient availability, temperature, pH, osmolarity, the presence of heavy metal(s), the concentration of inhibitor(s), stress, or a combination of the foregoing, as is known in the art. In some embodiments, the inducible or repressible promoters are inducible or repressible by metabolic factors, such as the level of certain carbon sources, the level of certain energy sources, the level of certain catabolites, or a combination of the foregoing as is known in the art.

In one embodiment, the promoter is one that is native to the host cell. For example, in some instances when Trichoderma reesei is the host, the promoter can be a native T. reesei promoter such as the cbh1 promoter which is deposited in GenBank under Accession Number 1786235. Other suitable non-limiting examples of promoters useful for fungal expression include, cbh2, egl1, egl2, egl3, egl4, egl5, xyn1, and xyn2, repressible acid phosphatase gene (phoA) promoter of P. chrysogenus (see e.g., Graessle et al., (1997) Appl. Environ. Microbiol., 63:753-756), glucose repressible PCK1 promoter (see e.g., Leuker et al., (1997), Gene, 192:235-240), maltose inducible, glucose-repressible MET3 promoter (see Liu et al., (2006), Eukary. Cell, 5:638-649), pKi promoter and cpc1 promoter. Other examples of useful promoters include promoters from A. awamori and A. niger glucoamylase genes (see e.g., Nunberg et al., (1984) Mol. Cell Biol. 15 4:2306-2315 and Boel et al., (1984) EMBO J. 3:1581-1585). Also, the promoters of the T. reesei xln1 gene may be useful (see e.g., EPA 137280A1).

DNA fragments which control transcriptional termination may also be derived from various genes native to a preferred production host cell. In certain embodiments, the inclusion of a termination control region is optional. In certain embodiments, the expression vector includes a termination control region derived from the preferred host cell.

Methods for transforming nucleic acids into filamentous fungi such as Aspergillus spp., e.g., A. oryzae or A. niger, H. grisea, H. insolens, and T. reesei, are well known in the art. A suitable procedure for transformation of Aspergillus host cells is described, for example, in EP238023.

A suitable procedure for transformation of Trichoderma host cells is described, for example, in Steiger et al 2011, Appl. Environ. Microbiol. 77:114421. Uptake of DNA into the host Trichoderma sp. strain is dependent upon the calcium ion concentration. Generally, between about 10 mM CaCl₂ and 50 mM CaCl₂ is used in an uptake solution. Besides the need for the calcium ion in the uptake solution, other compounds generally included are a buffering system such as TE buffer (10 Mm Tris, pH 7.4; 1 mM EDTA) or 10 mM MOPS, pH 6.0 buffer (morpholinepropanesulfonic acid) and polyethylene glycol (PEG). It is believed that the polyethylene glycol acts to fuse the cell membranes, thus permitting the contents of the medium to be delivered into the cytoplasm of the Trichoderma sp. strain and the plasmid DNA is transferred to the nucleus. This fusion frequently leaves multiple copies of the plasmid DNA integrated into the host chromosome.

Usually a suspension containing the Trichoderma sp. protoplasts or cells that have been subjected to a permeability treatment at a density of 10⁵ to 10⁷/mL, preferably 2×10⁶/mL are used in transformation. A volume of 100 μL of these protoplasts or cells in an appropriate solution (e.g., 1.2 M sorbitol; 50 mM CaCl₂) are mixed with the desired DNA. Generally, a high concentration of PEG is added to the uptake solution. From 0.1 to 1 volume of 25% PEG 4000 can be added to the protoplast suspension. However, it is preferable to add about 0.25 volumes to the protoplast suspension. Additives such as dimethyl sulfoxide, heparin, spermidine, potassium chloride and the like may also be added to the uptake solution and aid in transformation. Similar procedures are available for other fungal host cells. (see, e.g., U.S. Pat. Nos. 6,022,725 and 6,268,328, both of which are incorporated by reference).

Preferably, genetically stable transformants are constructed with vector systems whereby the nucleic acid encoding the phytase polypeptide or fragment thereof is stably integrated into a host strain chromosome. Transformants are then purified by known techniques.

After the expression vector is introduced into the cells, the transfected or transformed cells are cultured under conditions favoring expression of genes under control of the promoter sequences.

Generally, cells are cultured in a standard medium containing physiological salts and nutrients (see, e.g., Pourquie, J. et al., BIOCHEMISTRY AND GENETICS OF CELLULOSE DEGRADATION, eds. Aubert, J. P. et al., Academic Press, pp. 71-86, 1988 and IImen, M. et al., (1997) Appl. Environ. 63:1298-1306). Common commercially prepared media (e.g., Yeast Malt Extract (YM) broth, Luria Bertani (LB) broth and Sabouraud Dextrose (SD) broth also find use in the present invention.

Culture-conditions are also standard, (e.g., cultures are incubated at approximately 28° C. in appropriate medium in shake cultures or fermenters until desired levels of phytase expression are achieved). Preferred culture conditions for a given filamentous fungus are known in the art and may be found in the scientific literature and/or from the source of the fungi such as the American Type Culture Collection and Fungal Genetics Stock Center.

After fungal growth has been established, the cells are exposed to conditions effective to cause or permit the expression of a phytase and particularly a phytase as defined herein. In cases where a phytase coding sequence is under the control of an inducible promoter, the inducing agent (e.g., a sugar, metal salt or antimicrobial), is added to the medium at a concentration effective to induce phytase expression. An engineered phytase polypeptide or fragment thereof secreted from the host cells can be used, with minimal post-production processing, as a whole broth preparation.

The preparation of a spent whole fermentation broth of a recombinant microorganism can be achieved using any cultivation method known in the art resulting in the expression of an engineered phytase polypeptide or fragment thereof.

The term “spent whole fermentation broth” is defined herein as unfractionated contents of fermentation material that includes culture medium, extracellular proteins (e.g., enzymes), and cellular biomass, it is understood that the term “spent whole fermentation broth” also encompasses cellular biomass that has been lysed or permeabilized using methods well known in the art.

After fermentation, a fermentation broth is obtained, the microbial cells and various suspended solids, including residual raw fermentation materials, are removed by conventional separation techniques in order to obtain a phytase solution. Filtration, centrifugation, microfiltration, rotary vacuum drum filtration, ultrafiltration, centrifugation followed by ultra-filtration, extraction, or chromatography, or the like, are generally used.

It is possible to optionally recover the desired protein from the production host. In another aspect, an engineered phytase polypeptide or fragment thereof containing culture supernatant is obtained by using any of the methods known to those skilled in the art.

Examples of these techniques include, but are not limited to, affinity chromatography (Tilbeurgh et al., (1984) FEBS Lett. 16:215), ion-exchange chromatographic methods (Goyal et al., (1991) Biores. Technol. 36:37; Fliess et al., (1983) Eur. Appl. Microbiol. Biotechnol. 17:314; Bhikhabhai et al, (1984) J. Appl. Biochem. 6:336; and Ellouz et al., (1987) Chromatography 396:307), including ion-exchange using materials with high resolution power (Medve et al., (1998) J. Chromatography A 808:153), hydrophobic interaction chromatography (See, Tomaz and Queiroz, (1999) J. Chromatography A 865:123; two-phase partitioning (See, Brumbauer, et al., (1999) Bioseparation 7:287); ethanol precipitation; reverse phase HPLC, chromatography on silica or on a cation-exchange resin such as DEAE, chromatofocusing, SDS-PAGE, ammonium sulfate precipitation, and gel filtration (e.g., Sephadex G-75). The degree of purification desired will vary depending on the use of the engineered phytase polypeptide or fragment thereof. In some embodiments, purification will not be necessary.

On the other hand, it may be desirable to concentrate a solution containing an engineered phytase polypeptide or fragment thereof in order to optimize recovery. Use of unconcentrated solutions requires increased incubation time in order to collect the enriched or purified enzyme precipitate. The enzyme containing solution is concentrated using conventional concentration techniques until the desired enzyme level is obtained. Concentration of the enzyme containing solution may be achieved by any of the techniques discussed herein. Exemplary methods of enrichment and purification include but are not limited to rotary vacuum filtration and/or ultrafiltration.

In addition, concentration of the desired protein product may be performed using, e.g., a precipitation agent, such as a metal halide precipitation agent. The metal halide precipitation agent, sodium chloride, can also be used as a preservative. The metal halide precipitation agent is used in an amount effective to precipitate the engineered phytase polypeptide or fragment thereof. The selection of at least an effective amount and an optimum amount of metal halide effective to cause precipitation of the enzyme, as well as the conditions of the precipitation for maximum recovery including incubation time, pH, temperature and concentration of enzyme, will be readily apparent to one of ordinary skill in the art, after routine testing. Generally, at least about 5% w/v (weight/volume) to about 25% w/v of metal halide is added to the concentrated enzyme solution, and usually at least 8% w/v.

Another alternative way to precipitate the enzyme is to use organic compounds. Exemplary organic compound precipitating agents include: 4-hydroxybenzoic acid, alkali metal salts of 4-hydroxybenzoic acid, alkyl esters of 4-hydroxybenzoic acid, and blends of two or more of these organic compounds. The addition of the organic compound precipitation agents can take place prior to, simultaneously with or subsequent to the addition of the metal halide precipitation agent, and the addition of both precipitation agents, organic compound and metal halide, may be carried out sequentially or simultaneously. Generally, the organic precipitation agents are selected from the group consisting of alkali metal salts of 4-hydroxybenzoic acid, such as sodium or potassium salts, and linear or branched alkyl esters of 4-hydroxybenzoic acid, wherein the alkyl group contains from 1 to 12 carbon atoms, and blends of two or more of these organic compounds. Additional organic compounds also include but are not limited to 4-hydroxybenzoic acid methyl ester (named methyl PARABEN), 4-hydroxybenzoic acid propyl ester (named propyl PARABEN). For further descriptions, see, e.g., U.S. Pat. No. 5,281,526. Addition of the organic compound precipitation agent provides the advantage of high flexibility of the precipitation conditions with respect to pH, temperature, concentration, precipitation agent, protein concentration, and time of incubation. Generally, at least about 0.01% w/v and no more than about 0.3% w/v of organic compound precipitation agent is added to the concentrated enzyme solution.

After the incubation period, the enriched or purified enzyme is then separated from the dissociated pigment and other impurities and collected by conventional separation techniques, such as filtration, centrifugation, microfiltration, rotary vacuum filtration, ultrafiltration, press filtration, cross membrane microfiltration, cross flow membrane microfiltration, or the like. Further enrichment or purification of the enzyme precipitate can be obtained by washing the precipitate with water. For example, the enriched or purified enzyme precipitate is washed with water containing the metal halide precipitation agent, or with water containing the metal halide and the organic compound precipitation agents.

B. Methods for Preparing a Feed Additive Composition

Engineered phytase polypeptides or fragments thereof as described herein or a feed additive composition comprising such engineered phytase polypeptides or fragments thereof may be used as, or in the preparation of, a feed.

Thus, there is described a dried enzyme composition for use in animal feed comprising any of the engineered phytase polypeptides or fragment thereof as described herein.

There is also described a liquid enzyme composition for use in animal feed comprising any of the engineered phytase polypeptides or fragment thereof as described herein.

The terms “feed additive composition” and “enzyme composition” are used interchangeably herein.

The feed may be in the form of a solution or as a solid or as a semi-solid depending on the use and/or the mode of application and/or the mode of administration.

In a preferred embodiment, the enzyme or feed additive composition described herein is admixed with a feed component to form a feedstuff.

The term “feed component” as used herein means all or part of the feed. Part of the feed may mean one constituent of the feedstuff or more than one constituent of the feed, e.g. 2 or 3 or 4 or more.

In one embodiment, the term “feed component” encompasses a premix or premix constituents. Preferably, the feed may be a fodder, or a premix thereof, a compound feed, or a premix thereof. A feed additive composition may be admixed with a compound feed, a compound feed component or to a premix of a compound feed or to a fodder, a fodder component, or a premix of a fodder.

Fodder encompasses plants that have been cut. Furthermore, fodder includes silage, compressed and pelleted feeds, oils and mixed rations, and also sprouted grains and legumes.

Suitably a premix as referred to herein may be a composition composed of microingredients such as vitamins, minerals, chemical preservatives, antibiotics, fermentation products, and other essential ingredients. Premixes are usually compositions suitable for blending into commercial rations.

As used herein the term “contacted” refers to the indirect or direct application of any of the engineered phytase polypeptides or fragments thereof (or composition comprising any of the engineered phytase polypeptides or fragments thereof) to a product (e.g. the feed). Examples of application methods which may be used, include, but are not limited to, treating the product in a material comprising the feed additive composition, direct application by mixing the feed additive composition with the product, spraying the feed additive composition onto the product surface or dipping the product into a preparation of the feed additive composition. In one embodiment, the feed additive composition of the present invention is preferably admixed with the product (e.g. feedstuff). Alternatively, the feed additive composition may be included in the emulsion or raw ingredients of a feedstuff. For some applications, it is important that the composition is made available on or to the surface of a product to be affected/treated. This allows the composition to impart a performance benefit.

In some aspects, any of the engineered phytase polypeptides or fragments thereof can be used for the pre-treatment of food or feed. For example, the feed having 10-300% moisture is mixed and incubated with the engineered phytase polypeptides or fragments thereof at 5-80° C., preferably at 25-50° C., more preferably between 30-45° C. for l min to 72 hours under aerobic conditions or 1 day to 2 months under anaerobic conditions. The pre-treated material can be fed directly to the animals (so called liquid feeding). The pre-treated material can also be steam pelleted at elevated temperatures of 60-120° C. The engineered phytase polypeptides or fragments thereof can be impregnated to feed or food material by a vacuum coater.

Any of the engineered phytase polypeptides or fragments thereof described herein (or composition comprising such engineered phytase polypeptides or fragments thereof) may be applied to intersperse, coat and/or impregnate a product (e.g, feedstuff or raw ingredients of a feedstuff) with a controlled amount of said enzyme.

In another aspect, the feed additive composition can be homogenized to produce a powder. The powder may be mixed with other components known in the art. The powder, or mixture comprising the powder, may be forced through a die and the resulting strands are cut into suitable pellets of variable length.

Optionally, the pelleting step may include a steam treatment, or conditioning stage, prior to formation of the pellets. The mixture comprising the powder may be placed in a conditioner, e.g. a mixer with steam injection. The mixture is heated in the conditioner up to a specified temperature, such as from 60-100° C., typical temperatures would be 70° C., 80° C., 85° C., 90° C. or 95° C. The residence time can be variable from seconds to minutes. It will be understood that any of the engineered phytase polypeptides or fragments thereof (or composition comprising any of the engineered phytase polypeptides or fragments thereof) described herein are suitable for addition to any appropriate feed material.

In other embodiments, the granule may be introduced into a feed pelleting process wherein the feed pretreatment process may be conducted between 70° C. and 95° C. for up to several minutes, such as between 85° C. and 95° C.

In some embodiments, any of the engineered phytase polypeptides or fragments thereof can be present in the feed in the range of 1 ppb (parts per billion) to 10% (w/w) based on pure enzyme protein. In some embodiments, the engineered phytase polypeptides or fragments thereof are present in the feedstuff is in the range of 1-100 ppm (parts per million). A preferred dose can be 1-20 g of an engineered phytase polypeptide or fragment thereof per ton of feed product or feed composition or a final dose of 1-20 ppm engineered phytase polypeptide or fragment thereof in the final feed product.

Preferably, an engineered phytase polypeptide or fragment thereof is present in the feed should be at least about 50-10,000 FTU/kg corresponding to roughly 0.1 to 20 mg engineered phytase polypeptide or fragment thereof protein/kg.

Ranges can include, but are not limited to, any combination of the lower and upper ranges discussed above.

Formulations and/or preparations comprising any of the engineered phytase polypeptides or fragments thereof and compositions described herein may be made in any suitable way to ensure that the formulation comprises active phytase enzymes. Such formulations may be as a liquid, a dry powder or a granule which may be uncoated/unprotected or may involve the use of a thermoprotectant coating depending upon the processing conditions. As was noted above, the engineered phytase polypeptides and fragments thereof can be formulated inexpensively on a solid carrier without specific need for protective coatings and still maintain activity throughout the conditioning and pelleting process. A protective coating to provide additional thermostability when applied in a solid form can be beneficial for obtaining pelleting stability when required in certain regions where harsher conditions are used or if conditions warrant it, e.g., as in the case of super conditioning feed above 90° C.

Feed additive composition described herein can be formulated to a dry powder or granules as described in WO2007/044968 (referred to as TPT granules) or WO1997/016076 or WO1992/01.2645 (each of which is incorporated herein by reference).

In one embodiment the feed additive composition may be formulated to a granule for feed compositions comprising: a core; an active agent (for example, a phytase, such as any of the engineered phytase polypeptides disclosed herein); and at least one coating, the active agent of the granule retaining at least 50% activity, at least 60% activity, at least 70% activity, at least 80% activity after conditions selected from one or more of a) a feed pelleting process, b) a steam-heated feed pretreatment process, c) storage, d) storage as an ingredient in air unpelleted mixture, and e) storage as an ingredient in a feed base mix or a feed premix comprising at least one compound selected from trace minerals, organic acids, reducing sugars, vitamins, choline chloride, and compounds which result in an acidic or a basic feed base mix or feed premix.

With regard to the granule at least one coating may comprise a moisture hydrating material that constitutes at least 55% w/w of the granule; and/or at least one coating may comprise two coatings. The two coatings may be a moisture hydrating coating and a moisture barrier coating. In some embodiments, the moisture hydrating coating may be between 25% and 60% w/w of the granule and the moisture barrier coating may be between 2% and 15% w/w of the granule. The moisture hydrating coating may be selected from inorganic salts, sucrose, starch, and maltodextrin and the moisture barrier coating may be selected from polymers, gums, whey and starch.

In other embodiments, the granule may be introduced into a feed pelleting process wherein the feed pretreatment process may be conducted between 70° C. and 95° C. for up to several minutes, such as between 85° C. and 95° C.

The feed additive composition may be formulated to a granule for animal feed comprising: a core; an active agent, the active agent of the granule retaining at least 80% activity after storage and after a steam-heated pelleting process where the granule is an ingredient; a moisture barrier coating; and a moisture hydrating coating that is at least 25% w/w of the granule, the granule having a water activity of less than 0.5 prior to the steam-heated pelleting process.

The granule may have a moisture barrier coating selected from polymers and gums and the moisture hydrating material may be an inorganic salt. The moisture hydrating coating may be between 25% and 45% w/w of the granule and the moisture barrier coating may be between 2% and 10% w/w of the granule.

Alternatively, the composition is in a liquid formulation suitable for consumption preferably such liquid consumption contains one or more of the following: a buffer, salt, sorbitol and/or glycerol.

Also, the feed additive composition may be formulated by applying, e.g. spraying, the enzyme(s) onto a carrier substrate, such as ground wheat for example.

In one embodiment, the feed additive composition may be formulated as a premix. By way of example only the premix may comprise one or more feed components, such as one or more minerals and/or one or more vitamins.

In one embodiment a direct fed microbial (“DFM”) and/or an engineered phytase polypeptide or fragment thereof are formulated with at least one physiologically acceptable carrier selected from at least one of maltodextrin, limestone (calcium carbonate), cyclodextrin, wheat or a wheat component, sucrose, starch, Na2SO4, Talc, PVA, sorbitol, benzoate, sorbate, glycerol, sucrose, propylene glycol, 1,3-propane diol, glucose, parabens, sodium chloride, citrate, acetate, phosphate, calcium, metabisulfite, formate and mixtures thereof.

It should be noted that any of the engineered phytase polypeptides and fragments thereof may be useful in grain applications, e.g. processing of grains for non-food/feed application, e.g. ethanol production.

C. Methods for Improving Performance Metrics in an Animal

Also provided herein are methods for improving the nutritional value of an animal feed, wherein any of the engineered phytases or fragments thereof as described herein can be added to animal feed. In still another embodiment, the disclosure relates to a method comprising administering to an animal an effective amount of a composition comprising any of the engineered phytases or fragments thereof as described herein to increase performance of the animal on one or more metrics. This effective amount can be administered to the animal in one or more doses.

The phrase, an “effective amount” as used herein, refers to the amount of an active agent (such as, a phytase, e.g. any of the engineered phytase polypeptides disclosed herein) required to confer improved performance on an animal on one or more metrics, either alone or in combination with one or more other active agents (such as, without limitation, one or more additional enzyme(s), one or more DFM(s), one or more essential oils, etc.).

The term “animal performance” as used herein may be determined by any metric such as, without limitation, the feed efficiency and/or weight gain of the animal and/or by the feed conversion ratio and/or by the digestibility of a nutrient in a feed (e.g., amino acid digestibility or phosphorus digestibility) and/or digestible energy or metabolizable energy in a feed and/or by nitrogen retention and/or by animals' ability to avoid the negative effects of diseases or by the immune response of the subject.

Animal performance characteristics may include but are not limited to: body weight; weight gain; mass; body fat percentage; height; body fat distribution; growth; growth rate; egg size; egg weight; egg mass; egg laying rate; mineral absorption; mineral excretion, mineral retention; bone density; bone strength; feed conversion rate (FCR); average daily feed intake (ADFI); Average daily gain (ADG) retention and/or a secretion of any one or more of copper, sodium, phosphorous, nitrogen and calcium; amino acid retention or absorption; mineralization, bone mineralization carcass yield and carcass quality.

By “improved animal performance on one or more metric” it is meant that there is increased feed efficiency, and/or increased weight gain and/or reduced feed conversion ratio and/or improved digestibility of nutrients or energy in a feed and/or by improved nitrogen retention and/or by improved ability to avoid the negative effects of necrotic enteritis and/or by an improved immune response in the subject resulting from the use of feed comprising the feed additive composition described herein as compared to a feed which does not comprise said feed additive composition.

Preferably, by “improved animal performance” it is meant that there is increased feed efficiency and/or increased weight gain and/or reduced feed conversion ratio. As used herein, the term “feed efficiency” refers to the amount of weight gain in an animal that occurs when the animal is fed ad-libitum or a specified amount of food during a period of time. “An improvement in a metric” or “improved metric” as used herein, refers to an improvement in at least one of the parameters listed under the metrics in an animal definition.

By “increased feed efficiency” it is meant that the use of a feed additive composition according the present invention in feed results in an increased weight gain per unit of feed intake compared with an animal fed without said feed additive composition being present.

As used herein, the term “feed conversion ratio” refers to the amount of feed fed to an animal to increase the weight of the animal by a specified amount.

An improved feed conversion ratio means a lower feed conversion ratio.

By “lower feed conversion ratio” or “improved feed conversion ratio” it is meant that the use of a feed additive composition in feed results in a lower amount of feed being required to be fed to an animal to increase the weight of the animal by a specified amount compared to the amount of feed required to increase the weight of the animal by the same amount when the feed does not comprise said feed additive composition.

The improvement in performance parameters may be in respect to a control in which the feed used does not comprise a phytase.

The term Tibia ash refers to a quantification method for bone mineralization. This parameter gives indication if phosphorus is deficient (e.g. the content should be low in the phosphorus deficient negative control diets) or sufficient (e.g. the content in phytase treatments are comparable to a positive control diets that meeting phosphorus requirement in broilers)

The term “phosphorus deficient diet” refers to a diet in which the phosphorous level is not sufficient to satisfy the nutritional requirements of an animal, e.g., a feed formulated with phosphorus levels much lower than the recommended levels by the National Research Council (NRC) or broiler breeders. The animal feed contains lower levels of the mineral than required for optimal growth. If the diet lacks phosphorus, the calcium will also not be taken up by the animal. Excess Ca can lead to poor phosphorus (P) digestibility and contribute to the formation of insoluble mineral-phytate complexes. Both deficiency of P and Ca can cause reduced skeletal integrity, subnormal growth and ultimately weight loss.

The terms “mineralization” or “mineralization” encompass mineral deposition or release of minerals. Minerals may be deposited or released from the body of the animal. Minerals may be released from the feed. Minerals may include any minerals necessary in an animal diet, and may include calcium, copper, sodium, phosphorus, iron and nitrogen. In a preferred embodiment, use of the engineered phytase polypeptides or fragments thereof of the invention in a food or feed leads to increased calcium deposition in the body of the especially in the bones.

Nutrient digestibility as used herein means the fraction of a nutrient that disappears from the gastro-intestinal tract or a specified segment of the gastro-intestinal tract, e.g. the small intestine. Nutrient digestibility may be measured as the difference between what is administered to the subject and what comes out in the faeces of the subject, or between what is administered to the subject and what remains in the digesta on a specified segment of the gastro intestinal tract, e.g., the ileum.

Nutrient digestibility as used herein may be measured by the difference between the intake of a nutrient and the excreted nutrient by means of the total collection of excreta during a period of time; or with the use of an inert marker that is not absorbed by the animal, and allows the researcher calculating the amount of nutrient that disappeared in the entire gastro-intestinal tract or a segment of the gastro-intestinal tract. Such an inert marker may be titanium dioxide, chromic oxide or acid insoluble ash. Digestibility may be expressed as a percentage of the nutrient in the feed, or as mass units of digestible nutrient per mass units of nutrient in the feed.

Nutrient digestibility as used herein encompasses phosphorus digestibility, starch digestibility, fat digestibility, protein digestibility, and amino acid digestibility. Digestible phosphorus (P) can be defined as ileal digestible P which is the proportion of total P intake absorbed at the end of the ileum by an animal or the fecal digestible P which is the proportion of total P intake that is not excreted in the feces.

The term “survival” as used herein means the number of subjects remaining alive. The term “improved survival” is another way of saying “reduced mortality”.

The term “carcass yield” as used herein means the amount of carcass as a proportion of the live body weight, after a commercial or experimental process of slaughter. The term carcass means the body of an animal that has been slaughtered for food, with the head, entrails, part of the limbs, and feathers or skin removed. The term meat yield as used herein means the amount of edible meat as a proportion of the live body weight, or the amount of a specified meat cut as a proportion of the live body weight.

The terms “carcass quality” and “meat quality” are used interchangeably and refers to the compositional quality (lean to fat ratio) as well as palatability factors such as visual appearance, smell, firmness, juiciness, tenderness, and flavor. For example, producing poultry that does not have a “woody breast.” The woody breast is a quality issue stemming from a muscle abnormality in a small percentage of chicken meat in the U.S. This condition causes chicken breast meat to be hard to the touch and often pale in color with poor quality texture. Woody breast does not create any health or food safety concerns for people and the welfare of the chicken itself is not negatively impacted.

An “increased weight gain” refers to an animal having increased body weight on being fed feed comprising a feed additive composition compared with an animal being fed a feed without said feed additive composition being present.

In the present context, it is intended that the term “pet food” is understood to mean a food for a household animal such as, but not limited to, dogs, cats, gerbils, hamsters, chinchillas, fancy rats, guinea pigs; avian pets, such as canaries, parakeets, and parrots; reptile pets, such as turtles, lizards and snakes; and aquatic pets, such as tropical fish and frogs.

The terms “animal feed composition,” “feed”, “feedstuff,” and “fodder” are used interchangeably and can comprise one or more feed materials selected from the group comprising a) cereals, such as small grains (e.g., wheat, barley, rye, oats and combinations thereof) and/or large grains such as maize or sorghum; b) by products from cereals, such as corn gluten meal, Distillers Dried Grains with Solubles (DDGS) (particularly corn based Distillers Dried Grains with Solubles (cDDGS), wheat bran, wheat middlings, wheat shorts, rice bran, rice hulls, oat hulls, palm kernel, and citrus pulp; c) protein obtained from sources such as soya, sunflower, peanut, lupin, peas, fava beans, cotton, canola, fish meal, dried plasma protein, meat and bone meal, potato protein, whey, copra, sesame; d) oils and fats obtained from vegetable and animal sources; and/or e) minerals and vitamins.

In another embodiment, the disclosure relates to a method comprising administering to an animal an effective amount of a composition comprising an engineered phytase polypeptide or fragment thereof (such as any of the engineered phytase polypeptides disclosed herein) to increase average daily feed intake. In some embodiments, the average daily feed intake increases by any of about 10%, 15%, 20%, 25%, 30%, 35%, 40%, 45%, 50%, 55%, 60%, 65%, 70%, 75%, 80%, 85%, 90%, 95%, 100%, 105%, or 110%, inclusive of all values falling in between these percentages, relative to animals who are not administered one or more of the engineered phytase polypeptides or fragments thereof disclosed herein.

In another embodiment, the disclosure relates to a method comprising administering to an animal an effective amount of a composition comprising an engineered phytase polypeptide or fragment thereof (such as any of the engineered phytase polypeptides disclosed herein) to increase average daily weight gain. In some embodiments, the average daily weight gain increases by any of about 10%, 15%, 20%, 25%, 30%, 35%, 40%, 45%, 50%, 55%, 60%, 65%, 70%, 75%, 80%, 85%, 90%, 95%, 100%, 105%, or 110%, inclusive of all values falling in between these percentages, relative to animals who are not administered one or more of the engineered phytase polypeptides or fragments thereof disclosed herein.

In another embodiment, the disclosure relates to a method comprising administering to an animal an effective amount of a composition comprising an engineered phytase polypeptide or fragment thereof (such as any of the engineered phytase polypeptides disclosed herein) to increase total weight gain. In some embodiments, total weight gain increases by any of about 10%, 15%, 20%, 25%, 30%, 35%, 40%, 45%, 50%, 55%, 60%, 65%, 70%, 75%, 80%, 85%, 90%, 95%, 100%, 105%, or 110%, inclusive of all values falling in between these percentages, relative to animals who are not administered one or more of the engineered phytase polypeptides or fragments thereof disclosed herein.

In another embodiment, the disclosure relates to a method comprising administering to an animal an effective amount of a composition comprising an engineered phytase polypeptide or fragment thereof (such as any of the engineered phytase polypeptides disclosed herein) to increase feed conversion, which can be measured by either feed:gain or gain:feed. In some embodiments, feed conversion increases by any of about 10%, 15%, 20%, 25%, 30%, 35%, 40%, 45%, 50%, 55%, 60%, 65%, 70%, 75%, 80%, 85%, 90%, 95%, 100%, 105%, or 110%, inclusive of all values falling in between these percentages, relative to animals who are not administered one or more of the engineered phytase polypeptides or fragments thereof disclosed herein.

In another embodiment, the disclosure relates to a method comprising administering to an animal an effective amount of a composition comprising an engineered phytase polypeptide or fragment thereof (such as any of the engineered phytase polypeptides disclosed herein) to increase feed efficiency. In some embodiments, feed efficiency increases by any of about 10%, 15%, 20%, 25%, 30%, 35%, 40%, 45%, 50%, 55%, 60%, 65%, 70%, 75%, 80%, 85%, 90%, 95%, 100%, 105%, or 110%, inclusive of all values falling in between these percentages, relative to animals who are not administered one or more of the engineered phytase polypeptides or fragments thereof disclosed herein.

In another embodiment, the disclosure relates to a method comprising administering to an animal an effective amount of a composition comprising an engineered phytase polypeptide or fragment thereof (such as any of the engineered phytase polypeptides disclosed herein) to decrease feed conversion ratio (FCR). In some embodiments, FCR decreases by any of about 10%, 15%, 20%, 25%, 30%, 35%, 40%, 45%, 50%, 55%, 60%, 65%, 70%, 75%, 80%, 85%, 90%, 95%, or 100%, inclusive of all values falling in between these percentages, relative to animals who are not administered one or more of the engineered phytase polypeptides or fragments thereof disclosed herein.

The invention can be further understood by reference to the following examples, which are provided by way of illustration and are not meant to be limiting.

### EXAMPLES

### Example 1: Generation of Phytase Molecules

DNA manipulations to generate phytase encoding genes were carried out using molecular biology techniques known in the art. Polynucleotide fragments corresponding to the coding sequences of phytase variants were synthesized by a vendor (Twist BioScience, US) using preferred codons for the fungal expression host Trichoderma reseei (T. reesei). A DNA sequence encoding the signal sequence from the pep1 aspartate protease from T. reseei (SEQ ID NO: 7) artificially interrupted by a pep1 intron was introduced downstream of the T. reesei cbhI promoter in a suitable expression plasmid to ensure efficient secretion of phytase variants. Synthetic phytase gene fragments were cloned behind the region coding for the secretion signal of the resultant pI1c-AFP vector via Seamless Cloning and Assembly kit (Invitrogen, US) according to recommendations of the supplier. The final expression vector contains the T. reesei cbhI promoter and terminator regions driving the expression of phytase genes, the T. reesei pyr2 selection marker, and ˜1 kb long flanking sequences derived from the T. reesei genome which are required for targeted integration of the expression cassette. These plasmids were propagated in Escherichia coli TOP10 cells (Invitrogen, US), and after sequence verification purified plasmids served as templates to amplify the expression cassettes across the flanking regions.

Linear PCR fragments were transformed into a suitable T. reesei host strain using the polyethylene glycol (PEG)-protoplast method. DNA fragments were integrated at a desired locus in the fungus genome via homologous recombination techniques.

All high throughput transformations of phytase variants were performed robotically in a 24 well MTP format using Biomek robots (Beckman Coulter, USA). In brief, transformation mixtures containing approximately 1-2 μg of DNA, 10 pmol of the RNP complex and 5×10⁶ protoplasts in a total volume of 60-70 μL were incubated for 30 min on ice, treated with 200 μL of 25% PEG solution followed by dilution with equal volume of 1.2M sorbitol/10 mM Tris/10 mM CaCl₂ pH 7.5 solution and poured in 1 ml of 3% low melting agarose containing 1M sorbitol in minimal medium. After sufficient growth spores from pooled transformants of each variant were harvested and used for inoculation of liquid cultures.

For the expression of recombinant phytases, the transformed T. reseei strains were cultured as follows: 10⁵-10⁶ spores was used to inoculate 1 ml of production medium (37 g/L glucose, 1 g/L sophorose, 9 g/L casamino acids, 10 g/L (NH₄)₂SO₄, 4.5 g/L KH₂PO₄, 1 g/L MgSO₄*7H₂O, 1 g/L CaCl₂*2H₂O, 33 g/L PIPPS buffer (pH 5.5), 0.25% T. reesei trace elements (100%: 175 g/L citric acid (anhydrous), 200 g/L FeSO₄*7H₂O, 16 g/L ZnSO₄*7H₂O, 3.2 g/L CuSO₄*5H₂O, 1.4 g/L MnSO₄*H₂O, 0.8 g/L H₃BO₃) in 24 well MTPs. After 6 days of fermentation at 28° C. and 200 rpm with 80% humidity, the cultures were filtered by centrifugation using hydrophilic PVDF membranes to obtain clarified supernatants used for analysis of the recombinant phytase enzymes.

### Example 2: In Vitro Assays for Phytase Enzymes

Activity ratio between pH 3.5 and 5.5 on IP6 substrate: The phytases were assayed for phytase activity using IP6 substrate solution (Sodium Phytate from Rice, Shanghai A Z Import and Export, Zhejiang Orient Phytic Acid Co. Ltd #Z0201301181). For evaluation at pH 5.5, phytase enzyme samples at a targeted activity of 50 U/ml were serially diluted to a final concentration of 0.1 U/ml using 100 mM Na acetate buffer, 0.025% Tween-20 and 0.05 mM CaCl₂, pH 5.5 in 96-well MTP prior to analysis. 70 μL of the IP6 substrate (0.20 mM) in 100 mM Na acetate, 0.025% Tween 20 and 0.05 mM CaCl₂, pH 5.5 was added to a 96-well MTP and 10 μL aliquot of the diluted enzyme was added for a final volume of 80 μL.

For evaluation at pH 3.5, phytase enzyme samples at a targeted activity of 50 U/ml were serially diluted to a final concentration of 0.1 U/ml in 100 mM Na acetate buffer, 0.025% Tween-20 and 0.05 mM CaCl₂, pH 5.5 in 96-well MTP prior to analysis. 70 μL of the IP6 substrate (0.20 mM) prepared in 100 mM glycine buffer containing 0.025% Tween-20 and 0.05 mM CaCl₂, pH 3.5 was added to a 96-well MTP followed by 10 μL aliquot of the diluted enzyme for a final volume of 80 μL.

MTP reaction plates were incubated for 10 min at 25° C. in an iEMS shaker (Thermo Scientific) with continuous mixing (1400 rpm) and the reactions were stopped by addition of 170 μL of Pi Blue stop reagent (PiBlue™ Phosphate Assay Kit, POPB-DP, BioAsay Systems, US). The plates were mixed and sealed before incubated for color development for 30 min at 25° C. in an iEMS shaker (650 rpm). After incubation, the color formation was determined by measuring the absorbance at 620 nm on a plate reader (Spectramax, Molecular Devices). The activity in U/ml on IP6 substrate of each phytase sample was calculated based on a fitted standard curve of phytase protein with known activity (FTU) covering a range of 0-124 FTU/ml as the mean of three replicates.

Determination of melting temperature (Tm) by DSC: Differential scanning calorimetry (DSC) measurements were carried out using a MicroCal™ VP-Capillary DSC System (GE healthcare). DSC is a powerful analytical tool for characterizing the stability of proteins and other biomolecules. It measures the enthalpy (ΔH) and temperature (Tₘ) of thermally-induced structural transitions in solution: Phytase protein samples diluted to a final concentration of 0.3-0.5 mg/mL in 100 mM Na acetate buffer, pH 5.5 were prepared. 400 μL of these protein samples, as well as a reference containing an identical amount of protein-free buffer, were added to a 96-well plate. The plate was placed in the temperature controlled auto-sampler compartment kept at 10° C. The protein samples and the reference were scanned from 20 to 120° C. at a scan rate of 2° C. per minute. The melting temperature (Tm) was determined as the temperature at the peak maximum of the transition from the folded to unfolded state. Maximum variation in the Tm was ±0.2° C. The ORIGIN software package (MicroCal, GE Healthcare) was used for baseline subtraction and calculation of the Tm values.

### Example 3: Ratio of Activity (pH 3.5 vs 5.5) and Thermostability Evaluation of Phytase Enzymes

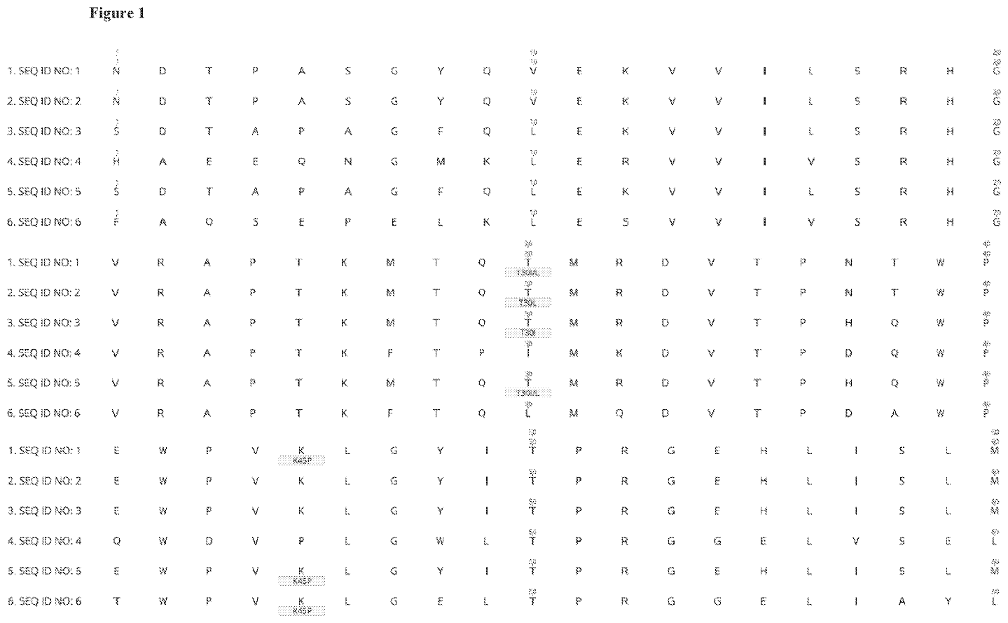

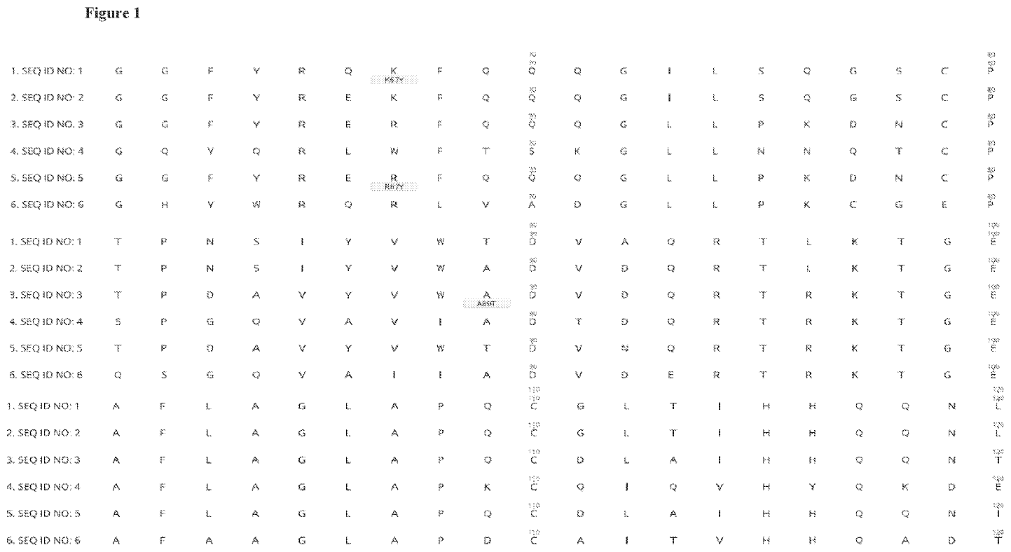

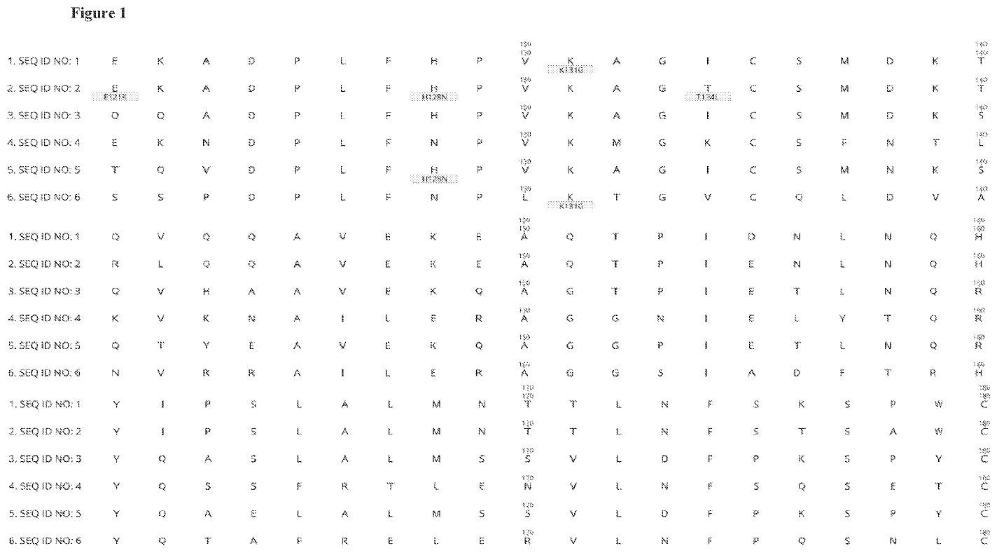

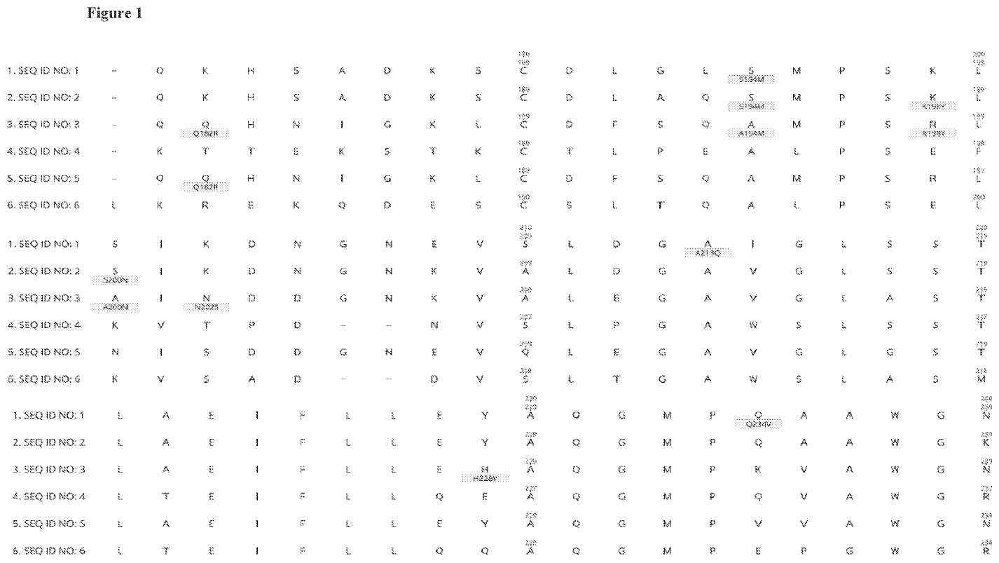

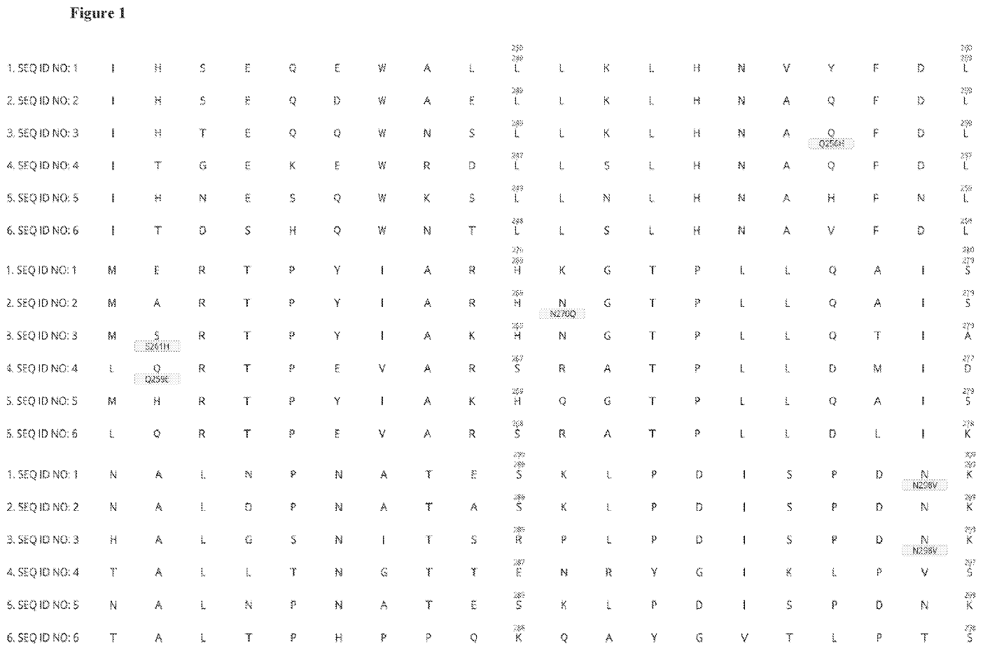

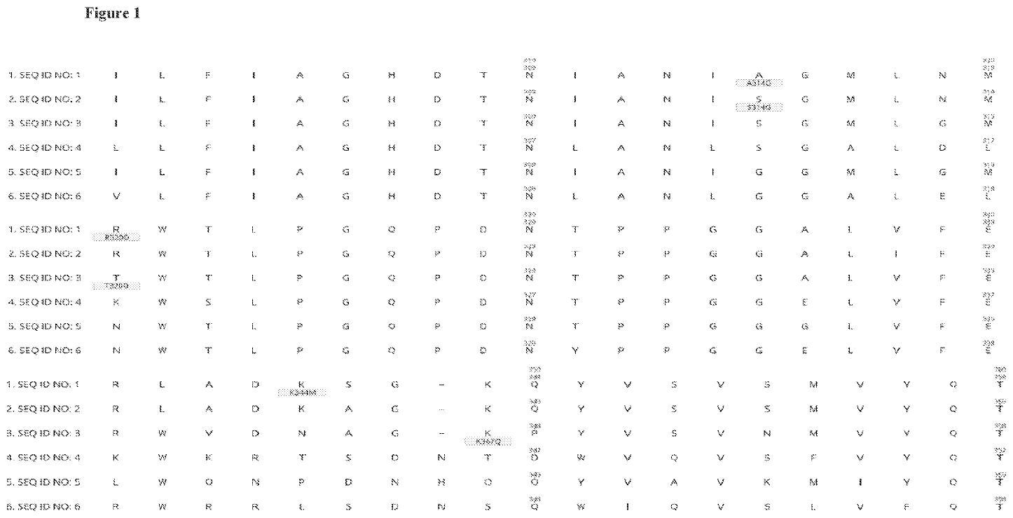

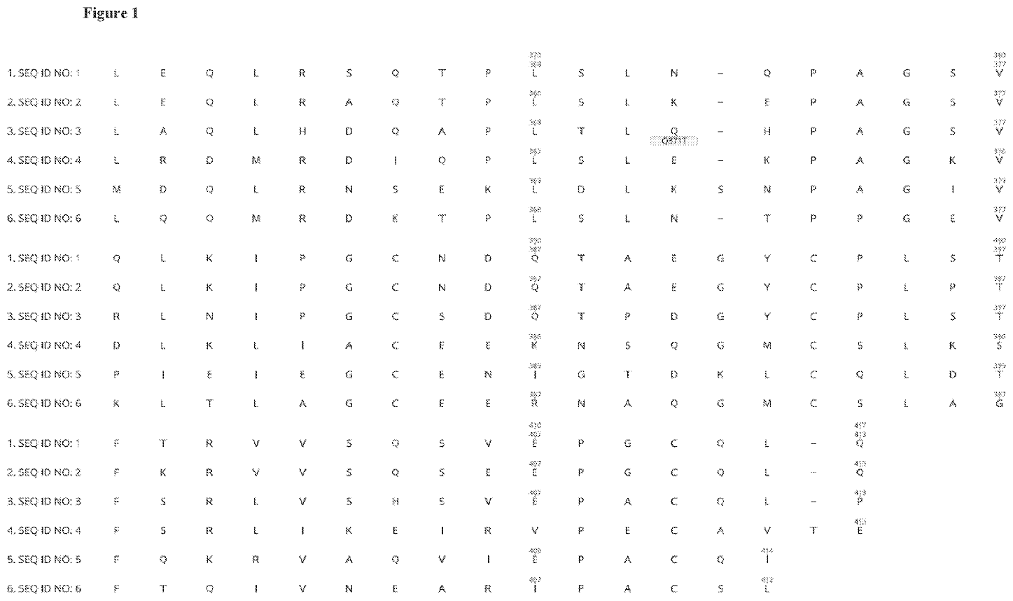

Samples of phytase polypeptides generated using the method described in Example 1 were evaluated for their phytase activity ratio at pH 3.5 versus 5.5 and for their thermostability, using methods described in Example 2. Amino acid substitutions were introduced by methods known in the art, described on Example 1, into various microbial HAP phytase sequences. The bacterial phytase sequences evaluated in the study include: SEQ ID NO: 1 (US8143046-0003), SEQ ID NO: 12 (US8101391-0002), SEQ ID NO: 3 (WO2010034835-0002), SEQ ID NO: 4 (AAS45884.1), SEQ ID NO: 5 (US8557555-0024), and SEQ ID NO: 6 (an E. coli phytase) and variants thereof, constructed as described in Example 1. The amino acid substitutions on the parent backbones are noted using the numbering corresponding to a multiple sequence alignment and SID 1 as reference sequence, FIG. 1. Table 1 provides the results for the phytase activity determined in the ferment at pH 5.5, the ratio of activity at pH 3.5 versus pH 5.5, and the thermostability (Tm) in ° C. measured by DSC. Selection is based on the relative performance index (PI) compared to parent backbone being larger than 1.00 and 1.0 for either thermostability or activity ratio respectively. In Table 1 the property is given in Selection criteria column as Thermostability (T) or activity ratio (A).

TABLE 1
Comparison of various substitutions across multiple bacterial
phytase backbones.

| | Phytase parent | | | | |
|---|---|---|---|---|---|
| | or variant | | Phytase | Ratio of | Tm |
| | (position | | Activity | activity at pH | (° C.) |
| Sample | # according to | Selection | at pH 5.5 | 3.5 versus 5.5 | by |
| ID | parent SID) | Criteria | (U/ml) | (PI) | DSC |
| SID1 | parent | | 6145 | 1.0 | 72.9 |
| SID1.v1 | T30I | T | 5268 | 0.9 | 73.3 |
| SID1.v2 | T30L | T | 4461 | 1.0 | 74.1 |
| SID1.v3 | K45P | T/A | 5685 | 1.1 | 74.7 |
| SID1.v4 | K67Y | A | 6607 | 1.1 | 72.2 |
| SID1.v5 | K131G | A | 7423 | 1.1 | ND |
| SID1.v6 | S194M | T | 4891 | 0.9 | 73.3 |
| SID1.v7 | A213Q | A | 2921 | 1.2 | ND |
| SID1.v8 | Q234V | A | 4471 | 1.2 | ND |
| SID1.v9 | N298V | A | 5188 | 1.1 | 72.9 |
| SID1.v10 | A314G | T/A | 5522 | 1.1 | 73.2 |
| SID1.v11 | R320D | A | 5022 | 1.2 | ND |
| SID1.v12 | K344M | A | 4969 | 1.1 | ND |
| SID2 | parent | | 7885 | 1.1 | ND |
| SID2.v1 | T30L | T/A | 6552 | 1.1 | ND |
| SID2.v2 | E121K | A | 6886 | 1.5 | ND |
| SID2.v3 | H128N | A | 4788 | 1.5 | ND |
| SID2.v4 | T134L | A | 7691 | 1.1 | ND |
| SID2.v5 | S194M | A | 6268 | 1.1 | ND |
| SID2.v6 | K198Y | A | 5684 | 1.1 | ND |
| SID2.v7 | S200N | A | 7248 | 1.2 | ND |
| SID2.v8 | N270Q | A | 6123 | 1.2 | ND |
| SID2.v9 | S314G | T/A | 10597 | 1.2 | ND |
| SID3 | parent | | 5620 | 1.0 | 71.0 |
| SID3.v1 | T30I | T | 6062 | 0.9 | 71.7 |
| SID3.v2 | A89T | T/A | 3691 | 1.2 | 72.1 |
| SID3.v3 | Q182R | T | 3466 | ND | 72.4 |
| SID3.v4 | A194M | T/A | 5211 | 1.6 | 72.3 |
| SID3.v5 | R198Y | A | 4829 | 1.2 | ND |
| SID3.v6 | A200N | A | 5516 | 1.2 | ND |
| SID3.v7 | N202S | T/A | 6072 | 1.1 | 71.7 |
| SID3.v8 | H228Y | T/A | 4433 | 1.3 | 73.0 |
| SID3.v9 | Q256H | T | 2191 | ND | 73.4 |
| SID3.v10 | S261H | T | 1596 | ND | 73.4 |
| SID3.v11 | N298V | T/A | 2001 | 1.5 | 72.2 |
| SID3.v12 | T320D | A | 5749 | 1.1 | ND |
| SID3.v13 | K347Q | A | 4898 | 1.4 | ND |
| SID3.v14 | Q371T | A | 4303 | 1.7 | ND |
| SID4 | parent | | 8152 | 1.0 | ND |
| SID4.v1 | Q259E | A | 5500 | 1.5 | ND |
| SID5 | parent | | 8745 | 1.0 | 87.5 |
| SID5.v1 | T30I | T | 8914 | 0.9 | 88.1 |
| SID5.v2 | T30L | T | 5589 | 1.0 | 88.8 |
| SID5.v3 | K45P | T | 7919 | 0.7 | 90.2 |
| SID5.v4 | R67Y | T | 8235 | 0.8 | 88.4 |
| SID5.v5 | H128N | A | 9134 | 1.2 | ND |
| SID5.v6 | Q182R | T | 6720 | 1.0 | 88.4 |
| SID6 | parent | | 7436 | 1.0 | 84.9 |
| SID6.v1 | D37Y | T | 8207 | 0.9 | 85.5 |
| SID6.v2 | K45P | T | 5514 | 1.0 | 85.9 |
| SID6.v3 | K13IG | A | 5579 | 1.6 | ND |

### Example 4: Sequence Comparison of Phytase Enzymes

The MAFFT alignment program in Geneious® version 10.2.4 was used for multiple sequence alignment of SEQ ID NO: 1 (US8143046-0003), SEQ ID NO: 12 (US8101391-0002), SEQ ID NO: 3 (WO2010034835-0002), SEQ ID NO: 4 (AAS45884.1), SEQ ID NO: 5 (US8557555-0024), and SEQ ID NO: 6 (a E. coli phytase) sequences, using default parameters and the alignment is shown on FIG. 1. Amino acid identity and similarities are highlighted in different shades, and the positions were amino acid substitutions were introduced with respect to parent phytases are shown.

SEQUENCES

NDTPASGYQVEKVVILSRHGVRAPTKMTQTMRDVTPNTWPEWPVKLGYIT
PRGEHLISLMGGFYRQKFQQQGILSQGSCPTPNSIYVWTDVAQRTLKTGE
AFLAGLAPQCGLTIHHQQNLEKADPLFHPVKAGICSMDKTQVQQAVEKEA
QTPIDNLNQHYIPSLALMNTTLNFSKSPWCQKHSADKSCDLGLSMPSKLS
IKDNGNEVSLDGAIGLSSTLAEIFLLEYAQGMPQAAWGNIHSEQEWALLL
KLHNVYFDLMERTPYIARHKGTPLLQAISNALNPNATESKLPDISPDNKI
LFIAGHDTNIANIAGMLNMRWTLPGQPDNTPPGGALVFERLADKSGKQYV
SVSMVYQTLEQLRSQTPLSLNQPAGSVQLKIPGCNDQTAEGYCPLSTFTR
VVSQSVEPGCQLQ (SEQ ID NO: 1)
NDTPASGYQVEKVVILSRHGVRAPTKMTQTMRDVTPNTWPEWPVKLGYIT
PRGEHLISLMGGFYREKFQQQGILSQGSCPTPNSIYVWADVDQRTLKTGE
AFLAGLAPQCGLTIHHQQNLEKADPLFHPVKAGTCSMDKTRLQQAVEKEA
QTPIENLNQHYIPSLALMNTTLNFSTSAWCQKHSADKSCDLAQSMPSKLS
IKDNGNKVALDGAVGLSSTLAEIFLLEYAQGMPQAAWGKIHSEQDWAELL
KLHNAQFDLMARTPYIARHNGTPLLQAISNALDPNATASKLPDISPDNKI
LFIAGHDTNIANISGMLNMRWTLPGQPDNTPPGGALIFERLADKAGKQYV
SVSMVYQTLEQLRAQTPLSLKEPAGSVQLKIPGCNDQTAEGYCPLPTFKR
VVSQSEEPGCQLQ (SEQ ID NO: 2)
SDTAPAGFQLEKVVILSRHGVRAPTKMTQTMRDVTPHQWPEWPVKLGYIT
PRGEHLISLMGGFYRERFQQQGLLPKDNCPTPDAVYVWADVDQRTRKTGE
AFLAGLAPQCDLAIHHQQNTQQADPLFHPVKAGICSMDKSQVHAAVEKQA
GTPIETLNQRYQASLALMSSVLDFPKSPYCQQHNIGKLCDFSQAMPSRLA
INDDGNKVALEGAVGLASTLAEIFLLEHAQGMPKVAWGNIHTEQQWNSLL
KLHNAQFDLMSRTPYIAKHNGTPLLQTIAHALGSNITSRPLPDISPDNKI
LFIAGHDTNIANISGMLGMTWTLPGQPDNTPPGGALVFERWVDNAGKPYV
SVNMVYQTLAQLHDQAPLTLQHPAGSVRLNIPGCSDQTPDGYCPLSTFSR
LVSHSVEPACQLP (SEQ ID NO: 3)
HAEEQNGMKLERVVIVSRHGVRAPTKFTPIMKDVTPDQWPQWDVPLGWLT
PRGGELVSELGQYQRLWFTSKGLLNNQTCPSPGQVAVIADTDQRTRKTGE
AFLAGLAPKCQIQVHYQKDEEKNDPLFNPVKMGKCSFNTLKVKNAILERA
GGNIELYTQRYQSSFRTLENVLNFSQSETCKTTEKSTKCTLPEALPSEFK
VTPDNVSLPGAWSLSSTLTEIFLLQEAQGMPQVAWGRITGEKEWRDLLSL
HNAQFDLLQRTPEVARSRATPLLDMIDTALLTNGTTENRYGIKLPVSLLF
IAGHDTNLANLSGALDLKWSLPGQPDNTPPGGELVFEKWKRTSDNTDWVQ
VSFVYQTLRDMRDIQPLSLEKPAGKVDLKLIACEEKNSQGMCSLKSFSRL
IKEIRVPECAVTE (SEQ ID NO: 4)
SDTAPAGFQLEKVVILSRHGVRAPTKMTQTMRDVTPHQWPEWPVKLGYIT
PRGEHLISLMGGFYRERFQQQGLLPKDNCPTPDAVYVWTDVNQRTRKTGE
AFLAGLAPQCDLAIHHQQNITQVDPLFHPVKAGICSMNKSQTYEAVEKQA
GGPIETLNQRYQAELALMSSVLDFPKSPYCQQHNIGKLCDFSQAMPSRLN
ISDDGNEVQLEGAVGLGSTLAEIFLLEYAQGMPVVAWGNIHNESQWKSLL
NLHNAHFNLMHRTPYIAKHQGTPLLQAISNALNPNATESKLPDISPDNKI
LFIAGHDTNIANIGGMLGMNWTLPGQPDNTPPGGGLVFELWQNPDNHQQY
VAVKMIYQTMDQLRNSEKLDLKSNPAGIVPIEIEGCENIGTDKLCQLDTF
QKRVAQVIEPACQI (SEQ ID NO: 5)
FAQSEPELKLESVVIVSRHGVRAPTKFTQLMQDVTPDAWPTWPVKLGELT
PRGGELIAYLGHYWRQRLVADGLLPKCGEPQSGQVAIIADVDERTRKTGE
AFAAGLAPDCAITVHHQADTSSPDPLFNPLKTGVCQLDVANVRRAILERA
GGSIADFTRHYQTAFRELERVLNFPQSNLCLKREKQDESCSLTQALPSEL
KVSADDVSLTGAWSLASMLTEIFLLQQAQGMPEPGWGRITDSHQWNTLLS
LHNAVFDLLQRTPEVARSRATPLLDLIKTALTPHPPQKQAYGVTLPTSVL
FIAGHDTNLANLGGALELNWTLPGQPDNYPPGGELVFERWRRLSDNSQWI
QVSLVFQTLQQMRDKTPLSLNTPPGEVKLTLAGCEERNAQGMCSLAGFTQ
IVNEARIPACSL (SEQ ID NO: 6)
MQTFGAFLVSFLAASGLAAA (SEQ ID NO: 7)

We claim:

1. An engineered phytase polypeptide or a fragment thereof comprising one or more substitutions selected from the group consisting of 30(L, I), 37Y, 45P, 67Y, 89T, 182R, 194M, 202S, 228Y, 256H, 261H, 298V, and 314G wherein the amino acid positions correspond to SEQ ID NO:1.

2. The polypeptide or fragment thereof of claim 1, further comprising one or more substitutions selected from the group consisting of 121K, 128N, 131G, 134L, 198Y, 200N, 213Q, 234V, 259E, 270Q, 298V, 320D, 344M, 347Q, and 371T.

3. The polypeptide or fragment thereof of claim 1 or claim 2, wherein the phytase polypeptide or a fragment thereof has at least 80% sequence identity to SEQ ID NO:1.

4. An engineered phytase polypeptide or a fragment thereof having at least 80% sequence identity to SEQ ID NO:2 and comprising one or more substitutions selected from the group consisting of 30L and 314G, wherein the amino acid positions correspond to SEQ ID NO:2.

5. The polypeptide or fragment thereof of claim 4, further comprising one or more substitutions selected from the group consisting of 121K, 128N, 134L, 194M, 198Y, 200N, and 270Q.

6. An engineered phytase polypeptide or a fragment thereof having at least 80% sequence identity to SEQ ID NO:3, and comprising one or more substitutions selected from the group consisting of 30I, 89T, 182R, 194M, 202S, 228Y, 256H, 261H, and 298V, wherein the amino acid positions correspond to SEQ ID NO:3.

7. The polypeptide or fragment thereof of claim 6, further comprising one or more substitutions selected from the group consisting of 198Y, 200N, 320D, 347Q, and 371T.

8. An engineered phytase polypeptide or a fragment thereof having at least 80% sequence identity to SEQ ID NO:4 comprising a 259E substitution, wherein the amino acid position corresponds to SEQ ID NO:4.

9. An engineered phytase polypeptide or a fragment thereof having at least 80% sequence identity to SEQ ID NO:5 comprising one or more substitutions selected from the group consisting of 30(I/L), 45P, 67Y, and 182R, wherein the amino acid position corresponds to SEQ ID NO:5.

10. The polypeptide or fragment thereof of claim 9, further comprising a 128N substitution.

11. An engineered phytase polypeptide or a fragment thereof having at least 80% sequence identity to SEQ ID NO:6 comprising one or more substitutions selected from the group consisting of 37Y and 45P, wherein the amino acid position corresponds to SEQ ID NO:6.

12. The polypeptide or fragment thereof of claim 11, further comprising a 131G substitution.

13. The polypeptide or fragment thereof any one of claims 1-12, wherein said one or more substitution increases a) thermostability; and/or b) ratio of activity at pH 3.5 versus pH 5.5 compared to phytase polypeptides that lack said one or more substitutions.

14. An animal feed, feedstuff, feed additive composition or premix comprising the polypeptide or fragment thereof of any one of claims 1-13, wherein the engineered phytase polypeptide or fragment thereof may be used (i) alone or (ii) in combination with a direct fed microbial comprising at least one bacterial strain or (iii) with at least one other enzyme or (iv) in combination with a direct fed microbial comprising at least one bacterial strain and at least one other enzyme, or (v) any of (i), (ii), (iii) or (iv) further comprising at least one other feed additive component and, optionally, the engineered phytase polypeptide or fragment thereof is present in an amount of at least about 0.1 g/ton feed.

15. A recombinant construct comprising a regulatory sequence functional in a production host operably linked to a nucleotide sequence encoding the engineered phytase polypeptide or fragment thereof of any one of claims 1-13.

16. The recombinant construct of claim 15, wherein the production host is selected from the group consisting of bacterial, fungi, yeast, plants and algae.

17. A method for producing an engineered phytase polypeptide or fragment thereof comprising:
a. transforming a production host with the recombinant construct of claim 16; and
b. culturing the production host of step (a) under conditions whereby the engineered phytase polypeptide or fragment thereof is produced.

18. The method of claim 17, further comprising (c) recovering the polypeptide or fragment thereof from the production host.

19. A phytase-containing culture supernatant obtained by the method of claim 17 or claim 18.

20. A polynucleotide sequence encoding the engineered phytase polypeptide or fragment thereof of any one of claims 1-13.

21. A dried enzyme composition for use in animal feed comprising the engineered phytase polypeptide or fragment thereof of any one of claims 1-13.

22. The dried enzyme composition of claim 21, wherein the dried enzyme composition is a granulated feed additive composition.

23. A liquid enzyme composition for use in animal feed comprising the engineered phytase polypeptide or fragment thereof of any one of claims 1-13.

24. A method for improving the nutritional value of an animal feed, wherein the engineered phytase or fragment thereof of any one of claims 1-13, is added to animal feed.

25. A method for improving animal performance on one or more metrics comprising administering an effective amount of a) the engineered phytase polypeptide of any one of claims 1-13; or b) the animal feed, feedstuff, feed additive composition or premix of claim 14 to the animal.

26. The method of claim 25, wherein the one or more metrics is selected from the group consisting of increased feed efficiency, increased weight gain, reduced feed conversion ratio, improved digestibility of nutrients or energy in a feed, improved nitrogen retention, improved ability to avoid the negative effects of necrotic enteritis, and improved immune response.

27. The method of claim 25 or claim 26, wherein the animal is a monogastric animal selected from the group consisting of swine and poultry.

28. The method of claim 27, wherein the swine is selected from the group consisting of piglets, growing pigs, and sows.

29. The method of claim 27, wherein the poultry is selected from the group consisting of turkeys, ducks, chickens, broiler chicks, layers, geese, pheasants, quail, and emus.

30. The method of claim 25 or claim 26, wherein the animal is a ruminant animal selected from the group consisting of cattle, young calves, goats, sheep, giraffes, bison, moose, elk, yaks, water buffalo, deer, reindeer, caribou, camels, alpacas, llamas.


SEQUENCE LISTING (7 sequences)

SEQ ID NO: 1 - protein, 413 aa, Artificial Sequence
  Synthesized sequence
NDTPASGYQVEKVVILSRHGVRAPTKMTQTMRDVTPNTWPEWPVKLGYITPRGEHLISLMGGFYRQKFQQQGILSQGSCPTPNSIYVWTDVAQRTLKTGEAFLAGLAPQCGLTIHHQQNLEKADPLFHPVKAGICSMDKTQVQQAVEKEAQTPIDNLNQHYIPSLALMNTTLNFSKSPWCQKHSADKSCDLGLSMPSKLSIKDNGNEVSLDGAIGLSSTLAEIFLLEYAQGMPQAAWGNIHSEQEWALLLKLHNVYFDLMERTPYIARHKGTPLLQAISNALNPNATESKLPDISPDNKILFIAGHDTNIANIAGMLNMRWTLPGQPDNTPPGGALVFERLADKSGKQYVSVSMVYQTLEQLRSQTPLSLNQPAGSVQLKIPGCNDQTAEGYCPLSTFTRVVSQSVEPGCQLQ

SEQ ID NO: 2 - protein, 413 aa, Artificial Sequence
  Synthesized sequence
NDTPASGYQVEKVVILSRHGVRAPTKMTQTMRDVTPNTWPEWPVKLGYITPRGEHLISLMGGFYREKFQQQGILSQGSCPTPNSIYVWADVDQRTLKTGEAFLAGLAPQCGLTIHHQQNLEKADPLFHPVKAGTCSMDKTRLQQAVEKEAQTPIENLNQHYIPSLALMNTTLNFSTSAWCQKHSADKSCDLAQSMPSKLSIKDNGNKVALDGAVGLSSTLAEIFLLEYAQGMPQAAWGKIHSEQDWAELLKLHNAQFDLMARTPYIARHNGTPLLQAISNALDPNATASKLPDISPDNKILFIAGHDTNIANISGMLNMRWTLPGQPDNTPPGGALIFERLADKAGKQYVSVSMVYQTLEQLRAQTPLSLKEPAGSVQLKIPGCNDQTAEGYCPLPTFKRVVSQSEEPGCQLQ

SEQ ID NO: 3 - protein, 413 aa, Artificial Sequence
  Synthesized sequence
SDTAPAGFQLEKVVILSRHGVRAPTKMTQTMRDVTPHQWPEWPVKLGYITPRGEHLISLMGGFYRERFQQQGLLPKDNCPTPDAVYVWADVDQRTRKTGEAFLAGLAPQCDLAIHHQQNTQQADPLFHPVKAGICSMDKSQVHAAVEKQAGTPIETLNQRYQASLALMSSVLDFPKSPYCQQHNIGKLCDFSQAMPSRLAINDDGNKVALEGAVGLASTLAEIFLLEHAQGMPKVAWGNIHTEQQWNSLLKLHNAQFDLMSRTPYIAKHNGTPLLQTIAHALGSNITSRPLPDISPDNKILFIAGHDTNIANISGMLGMTWTLPGQPDNTPPGGALVFERWVDNAGKPYVSVNMVYQTLAQLHDQAPLTLQHPAGSVRLNIPGCSDQTPDGYCPLSTFSRLVSHSVEPACQLP

SEQ ID NO: 4 - protein, 413 aa, Artificial Sequence
  Synthesized sequence
HAEEQNGMKLERVVIVSRHGVRAPTKFTPIMKDVTPDQWPQWDVPLGWLTPRGGELVSELGQYQRLWFTSKGLLNNQTCPSPGQVAVIADTDQRTRKTGEAFLAGLAPKCQIQVHYQKDEEKNDPLFNPVKMGKCSFNTLKVKNAILERAGGNIELYTQRYQSSFRTLENVLNFSQSETCKTTEKSTKCTLPEALPSEFKVTPDNVSLPGAWSLSSTLTEIFLLQEAQGMPQVAWGRITGEKEWRDLLSLHNAQFDLLQRTPEVARSRATPLLDMIDTALLTNGTTENRYGIKLPVSLLFIAGHDTNLANLSGALDLKWSLPGQPDNTPPGGELVFEKWKRTSDNTDWVQVSFVYQTLRDMRDIQPLSLEKPAGKVDLKLIACEEKNSQGMCSLKSFSRLIKEIRVPECAVTE

SEQ ID NO: 5 - protein, 414 aa, Artificial Sequence
  Synthesized sequence
SDTAPAGFQLEKVVILSRHGVRAPTKMTQTMRDVTPHQWPEWPVKLGYITPRGEHLISLMGGFYRERFQQQGLLPKDNCPTPDAVYVWTDVNQRTRKTGEAFLAGLAPQCDLAIHHQQNITQVDPLFHPVKAGICSMNKSQTYEAVEKQAGGPIETLNQRYQAELALMSSVLDFPKSPYCQQHNIGKLCDFSQAMPSRLNISDDGNEVQLEGAVGLGSTLAEIFLLEYAQGMPVVAWGNIHNESQWKSLLNLHNAHFNLMHRTPYIAKHQGTPLLQAISNALNPNATESKLPDISPDNKILFIAGHDTNIANIGGMLGMNWTLPGQPDNTPPGGGLVFELWQNPDNHQQYVAVKMIYQTMDQLRNSEKLDLKSNPAGIVPIEIEGCENIGTDKLCQLDTFQKRVAQVIEPACQI

SEQ ID NO: 6 - protein, 412 aa, Artificial Sequence
  Synthesized sequence
FAQSEPELKLESVVIVSRHGVRAPTKFTQLMQDVTPDAWPTWPVKLGELTPRGGELIAYLGHYWRQRLVADGLLPKCGEPQSGQVAIIADVDERTRKTGEAFAAGLAPDCAITVHHQADTSSPDPLFNPLKTGVCQLDVANVRRAILERAGGSIADFTRHYQTAFRELERVLNFPQSNLCLKREKQDESCSLTQALPSELKVSADDVSLTGAWSLASMLTEIFLLQQAQGMPEPGWGRITDSHQWNTLLSLHNAVFDLLQRTPEVARSRATPLLDLIKTALTPHPPQKQAYGVTLPTSVLFIAGHDTNLANLGGALELNWTLPGQPDNYPPGGELVFERWRRLSDNSQWIQVSLVFQTLQQMRDKTPLSLNTPPGEVKLTLAGCEERNAQGMCSLAGFTQIVNEARIPACSL

SEQ ID NO: 7 - protein, 20 aa, Artificial Sequence
  Synthesized sequence
MQTFGAFLVSFLAASGLAAA